SEQUENCE LISTING (65 sequences)

SEQ ID NO: 1 - DNA, 2362 bp, synthetic construct
  source 1..2362: mol_type = other DNA; note = muPAR.h28z tNGFR
atggccctgccagtaacggctctgctgctgccacttgctctgctcctccatgcagccaggcctgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccgatgggggcaggtgccactggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagccgcgccaagcgctcgggttcgggta

SEQ ID NO: 2 - protein, 788 aa, synthetic construct
  source 1..788: note = muPAR.h28z tNGFR
MALPVTALLLPLALLLHAARPEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSRAKRSGSGX

SEQ ID NO: 3 - DNA, 2373 bp, synthetic construct
  source 1..2373: mol_type = other DNA; note = huPAR.h28z tNGFR
atggccctgccagtaacggctctgctgctgccacttgctctgctcctccatgcagccaggcctcaggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctgacatcgtgctgacccagagcccagctagcttggcagtgagcctgggacagagggctaccatcagctgcagagcttcagagagcgtggacagctacggaaacagcttcatgcactggtaccagcagaagccaggacagccacctaagctgctgatctaccgggctagcaacctgaagtccggaatccctgctcggtttagcggaagcggtagcggcaccgacttcaccctgacaatcaacccagtggaggccgacgatgtggcaacctactgctgtcagcagagcaacgaggacccatggaccttcggcggtggaaccaaactggagatcaagagaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccgatgggggcaggtgccactggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagccgcgccaagcgctcgggttcgggt

SEQ ID NO: 4 - protein, 791 aa, synthetic construct
  source 1..791: note = huPAR.h28z tNGFR
MALPVTALLLPLALLLHAARPQVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSDIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSRAKRSGSG

SEQ ID NO: 5 - DNA, 2625 bp, synthetic construct
  source 1..2625: mol_type = other DNA; note = huPAR.h28z mCherry
atggccctgccagtaacggctctgctgctgccacttgctctgctcctccatgcagccaggcctcaggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctgacatcgtgctgacccagagcccagctagcttggcagtgagcctgggacagagggctaccatcagctgcagagcttcagagagcgtggacagctacggaaacagcttcatgcactggtaccagcagaagccaggacagccacctaagctgctgatctaccgggctagcaacctgaagtccggaatccctgctcggtttagcggaagcggtagcggcaccgacttcaccctgacaatcaacccagtggaggccgacgatgtggcaacctactgctgtcagcagagcaacgaggacccatggaccttcggcggtggaaccaaactggagatcaagagaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccgatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaactccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtccgagggcactaaggcagttaccaagtacactagctctaaggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 6 - protein, 874 aa, synthetic construct
  source 1..874: note = huPAR.h28z mCherry
MALPVTALLLPLALLLHAARPQVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSDIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMPEPSKSAPAPKKGSKKAITKAQKKDGKKRKRSRKESYSIYVYKVLKQVHPDTGISSKAMGIMNSFVNDIFERIAGEASRLAHYNKRSTITSREIQTAVRLLLPGELAKHAVSEGTKAVTKYTSSKDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 7 - DNA, 2382 bp, synthetic construct
  source 1..2382: mol_type = other DNA; note = muPAR.m28z tNGFR
atggccagccccctgaccaggttcctgagcctgaacctgctgctgctgggcgagagcatcatcgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcgaacagaaactgattagcgaagaagatctgattgaatttatgtatccgccgccgtatctggataacgaacgcagcaacggcaccattattcatattaaagaaaaacatctgtgccatacccagagcagcccgaaactgttttgggcgctggtggtggtggcgggcgtgctgttttgctatggcctgctggtgaccgtggcgctgtgcgtgatttggaccaacagccgccgcaaccgcctgctgcagagcgattatatgaacatgaccccgcgccgcccgggcctgacccgcaaaccgtatcagccgtatgcgccggcgcgcgattttgcggcgtatcgcccgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccgatgggggcaggtgccactggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagccgcgccaagcgctcgggttcgggt

SEQ ID NO: 8 - protein, 794 aa, synthetic construct
  source 1..794: note = muPAR.m28z tNGFR
MASPLTRFLSLNLLLLGESIIEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKREQKLISEEDLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSRAKRSGSG

SEQ ID NO: 9 - DNA, 2613 bp, synthetic construct
  source 1..2613: mol_type = other DNA; note = muPAR.h28z mCherry
atggccctgccagtaacggctctgctgctgccacttgctctgctcctccatgcagccaggcctgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccgatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaactccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtccgagggcactaaggcagttaccaagtacactagctctaaggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 10 - protein, 870 aa, synthetic construct
  source 1..870: note = muPAR.h28z mCherry
MALPVTALLLPLALLLHAARPEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMPEPSKSAPAPKKGSKKAITKAQKKDGKKRKRSRKESYSIYVYKVLKQVHPDTGISSKAMGIMNSFVNDIFERIAGEASRLAHYNKRSTITSREIQTAVRLLLPGELAKHAVSEGTKAVTKYTSSKDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 11 - DNA, 2238 bp, synthetic construct
  source 1..2238: mol_type = other DNA; note = muPAR.m28z mCherry
atggccagccccctgaccaggttcctgagcctgaacctgctgctgctgggcgagagcatcatcgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcgaacagaaactgattagcgaagaagatctgattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcgcgaccaactttagcctgctgaaacaggcgggcgatgtggaagaaaacccgggcccggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 12 - protein, 745 aa, synthetic construct
  source 1..745: note = muPAR.m28z mCherry
MASPLTRFLSLNLLLLGESIIEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKREQKLISEEDLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 13 - DNA, 2484 bp, synthetic construct
  source 1..2484: mol_type = other DNA; note = muPAR.m28z APOE2
atggccagccccctgaccaggttcctgagcctgaacctgctgctgctgggcgagagcatcatcgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcgaacagaaactgattagcgaagaagatctgattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcgcgaccaactttagcctgctgaaacaggcgggcgatgtggaagaaaacccgggcccgatgaaagttttgtgggccgctttgttggtaacgttcttggcaggctgtcaggcgaaggttgaacaagcagtcgaaacggagccagagccagagctccgacagcagaccgaatggcaatctggtcaaaggtgggaacttgcgttgggccgattttgggattaccttagatgggtgcagacactttcagaacaggttcaggaggaattgcttagctcacaggtaactcaggagttgcgcgcacttatggacgagacgatgaaagaactcaaggcgtacaagagcgagctggaagagcagctcacacctgtagctgaagaaacacgcgcacggttgtctaaagaactccaggctgctcaggcccgcttgggagcagatatggaggacgtctgtggaagactcgtccagtatcggggcgaggtgcaggccatgttgggacaaagtacggaagagcttcgggtaagattggcaagccacctcaggaaactgagaaagagactcctgagagacgcggatgacctgcagaaatgtcttgcagtgtaccaagctggagctcgcgaaggcgctgaacggggactgagtgcgattagagaacgattgggccctcttgttgaacaggggagggttagagcggcgactgtcggttctctggcagggcagcctctgcaagagcgcgctcaagcttggggtgaacgccttagagcccgaatggaagagatgggctctcggacccgagatcgacttgatgaggtgaaggagcaagtggcggaagttcgagctaagctggaggaacaggcccaacaaatccgactccaagccgaggcttttcaagcaaggctgaaaagctggtttgaacccttggtcgaagacatgcagcgccagtgggcgggattggttgaaaaagtccaagccgcggttggcacgtccgccgcccccgtgccaagtgacaatcactaa

SEQ ID NO: 14 - protein, 827 aa, synthetic construct
  source 1..827: note = muPAR.m28z APOE2
MASPLTRFLSLNLLLLGESIIEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKREQKLISEEDLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 15 - DNA, 2502 bp, synthetic construct
  source 1..2502: mol_type = other DNA; note = huPAR.h28z APOE2
atggccctgccagtaacggctctgctgctgccacttgctctgctcctccatgcagccaggcctcaggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctgacatcgtgctgacccagagcccagctagcttggcagtgagcctgggacagagggctaccatcagctgcagagcttcagagagcgtggacagctacggaaacagcttcatgcactggtaccagcagaagccaggacagccacctaagctgctgatctaccgggctagcaacctgaagtccggaatccctgctcggtttagcggaagcggtagcggcaccgacttcaccctgacaatcaacccagtggaggccgacgatgtggcaacctactgctgtcagcagagcaacgaggacccatggaccttcggcggtggaaccaaactggagatcaagagaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccggaacagaaactgattagcgaagaagatctgatgaaagttttgtgggccgctttgttggtaacgttcttggcaggctgtcaggcgaaggttgaacaagcagtcgaaacggagccagagccagagctccgacagcagaccgaatggcaatctggtcaaaggtgggaacttgcgttgggccgattttgggattaccttagatgggtgcagacactttcagaacaggttcaggaggaattgcttagctcacaggtaactcaggagttgcgcgcacttatggacgagacgatgaaagaactcaaggcgtacaagagcgagctggaagagcagctcacacctgtagctgaagaaacacgcgcacggttgtctaaagaactccaggctgctcaggcccgcttgggagcagatatggaggacgtctgtggaagactcgtccagtatcggggcgaggtgcaggccatgttgggacaaagtacggaagagcttcgggtaagattggcaagccacctcaggaaactgagaaagagactcctgagagacgcggatgacctgcagaaatgtcttgcagtgtaccaagctggagctcgcgaaggcgctgaacggggactgagtgcgattagagaacgattgggccctcttgttgaacaggggagggttagagcggcgactgtcggttctctggcagggcagcctctgcaagagcgcgctcaagcttggggtgaacgccttagagcccgaatggaagagatgggctctcggacccgagatcgacttgatgaggtgaaggagcaagtggcggaagttcgagctaagctggaggaacaggcccaacaaatccgactccaagccgaggcttttcaagcaaggctgaaaagctggtttgaacccttggtcgaagacatgcagcgccagtgggcgggattggttgaaaaagtccaagccgcggttggcacgtccgccgcccccgtgccaagtgacaatcactaa

SEQ ID NO: 16 - protein, 833 aa, synthetic construct
  source 1..833: note = huPAR.h28z APOE2
MALPVTALLLPLALLLHAARPQVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSDIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPEQKLISEEDLMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 17 - DNA, 3279 bp, synthetic construct
  source 1..3279: mol_type = other DNA; note = miniXon huPAR.h28z mCherry
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcccgagagatctggcagcggagagggcagaggaagtcttctaacatgcggtgacgtggaggagaatcccggccctaggctcgagatggccctgccagtaacggctctgctgctgccacttgctctgctcctccatgcagccaggcctcaggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctgacatcgtgctgacccagagcccagctagcttggcagtgagcctgggacagagggctaccatcagctgcagagcttcagagagcgtggacagctacggaaacagcttcatgcactggtaccagcagaagccaggacagccacctaagctgctgatctaccgggctagcaacctgaagtccggaatccctgctcggtttagcggaagcggtagcggcaccgacttcaccctgacaatcaacccagtggaggccgacgatgtggcaacctactgctgtcagcagagcaacgaggacccatggaccttcggcggtggaaccaaactggagatcaagagaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccgatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaactccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtccgagggcactaaggcagttaccaagtacactagctctaaggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 18 - protein, 1077 aa, synthetic construct
  source 1..1077: note = miniXon huPAR.h28z mCherry
FLYNLTLQRATGISFAILGNFSGKFSRYHLLKFVNYISCLVSCAGNVFHLQNRNCVKFQILDEIQSYKGTNVYKIKTFSCHLQIHFVNPSATMQEVHDCVEDIILLRFNSEYLMWYILNVVLRGSLTDHTAFTPGFLYFRRGFTMVARLGSGCLSLFFGDLGTKQQEIVVSRGEDPERSGSGEGRGSLLTCGDVEENPGPRLEMALPVTALLLPLALLLHAARPQVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSDIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMPEPSKSAPAPKKGSKKAITKAQKKDGKKRKRSRKESYSIYVYKVLKQVHPDTGISSKAMGIMNSFVNDIFERIAGEASRLAHYNKRSTITSREIQTAVRLLLPGELAKHAVSEGTKAVTKYTSSKDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 19 - DNA, 2889 bp, synthetic construct
  source 1..2889: mol_type = other DNA; note = miniXon muPAR.m28z mCherry
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcccgagagatctggcagcggagagggcagaggaagtcttctaacatgcggtgacgtggaggagaatcccggccctaggctcgagatggccagccccctgaccaggttcctgagcctgaacctgctgctgctgggcgagagcatcatcgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcgaacagaaactgattagcgaagaagatctgattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcgcgaccaactttagcctgctgaaacaggcgggcgatgtggaagaaaacccgggcccggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 20 - protein, 948 aa, synthetic construct
  source 1..948: note = miniXon muPAR.m28z mCherry
FLYNLTLQRATGISFAILGNFSGKFSRYHLLKFVNYISCLVSCAGNVFHLQNRNCVKFQILDEIQSYKGTNVYKIKTFSCHLQIHFVNPSATMQEVHDCVEDIILLRFNSEYLMWYILNVVLRGSLTDHTAFTPGFLYFRRGFTMVARLGSGCLSLFFGDLGTKQQEIVVSRGEDPERSGSGEGRGSLLTCGDVEENPGPRLEMASPLTRFLSLNLLLLGESIIEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKREQKLISEEDLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 21 - DNA, 3138 bp, synthetic construct
  source 1..3138: mol_type = other DNA; note = miniXon muPAR.m28z APOE2
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcccgagagatctggcagcggagagggcagaggaagtcttctaacatgcggtgacgtggaggagaatcccggccctaggctcgagatggccagccccctgaccaggttcctgagcctgaacctgctgctgctgggcgagagcatcatcgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcgaacagaaactgattagcgaagaagatctgattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcgcgaccaactttagcctgctgaaacaggcgggcgatgtggaagaaaacccgggcccgatgaaagttttgtgggccgctttgttggtaacgttcttggcaggctgtcaggcgaaggttgaacaagcagtcgaaacggagccagagccagagctccgacagcagaccgaatggcaatctggtcaaaggtgggaacttgcgttgggccgattttgggattaccttagatgggtgcagacactttcagaacaggttcaggaggaattgcttagctcacaggtaactcaggagttgcgcgcacttatggacgagacgatgaaagaactcaaggcgtacaagagcgagctggaagagcagctcacacctgtagctgaagaaacacgcgcacggttgtctaaagaactccaggctgctcaggcccgcttgggagcagatatggaggacgtctgtggaagactcgtccagtatcggggcgaggtgcaggccatgttgggacaaagtacggaagagcttcgggtaagattggcaagccacctcaggaaactgagaaagagactcctgagagacgcggatgacctgcagaaatgtcttgcagtgtaccaagctggagctcgcgaaggcgctgaacggggactgagtgcgattagagaacgattgggccctcttgttgaacaggggagggttagagcggcgactgtcggttctctggcagggcagcctctgcaagagcgcgctcaagcttggggtgaacgccttagagcccgaatggaagagatgggctctcggacccgagatcgacttgatgaggtgaaggagcaagtggcggaagttcgagctaagctggaggaacaggcccaacaaatccgactccaagccgaggcttttcaagcaaggctgaaaagctggtttgaacccttggtcgaagacatgcagcgccagtgggcgggattggttgaaaaagtccaagccgcggttggcacgtccgccgcccccgtgccaagtgacaatcactaa

SEQ ID NO: 22 - protein, 1030 aa, synthetic construct
  source 1..1030: note = miniXon muPAR.m28z APOE2
FLYNLTLQRATGISFAILGNFSGKFSRYHLLKFVNYISCLVSCAGNVFHLQNRNCVKFQILDEIQSYKGTNVYKIKTFSCHLQIHFVNPSATMQEVHDCVEDIILLRFNSEYLMWYILNVVLRGSLTDHTAFTPGFLYFRRGFTMVARLGSGCLSLFFGDLGTKQQEIVVSRGEDPERSGSGEGRGSLLTCGDVEENPGPRLEMASPLTRFLSLNLLLLGESIIEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKREQKLISEEDLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 23 - DNA, 3156 bp, synthetic construct
  source 1..3156: mol_type = other DNA; note = miniXon huPAR.h28z APOE2
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcccgagagatctggcagcggagagggcagaggaagtcttctaacatgcggtgacgtggaggagaatcccggccctaggctcgagatggccctgccagtaacggctctgctgctgccacttgctctgctcctccatgcagccaggcctcaggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctgacatcgtgctgacccagagcccagctagcttggcagtgagcctgggacagagggctaccatcagctgcagagcttcagagagcgtggacagctacggaaacagcttcatgcactggtaccagcagaagccaggacagccacctaagctgctgatctaccgggctagcaacctgaagtccggaatccctgctcggtttagcggaagcggtagcggcaccgacttcaccctgacaatcaacccagtggaggccgacgatgtggcaacctactgctgtcagcagagcaacgaggacccatggaccttcggcggtggaaccaaactggagatcaagagaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccggaacagaaactgattagcgaagaagatctgatgaaagttttgtgggccgctttgttggtaacgttcttggcaggctgtcaggcgaaggttgaacaagcagtcgaaacggagccagagccagagctccgacagcagaccgaatggcaatctggtcaaaggtgggaacttgcgttgggccgattttgggattaccttagatgggtgcagacactttcagaacaggttcaggaggaattgcttagctcacaggtaactcaggagttgcgcgcacttatggacgagacgatgaaagaactcaaggcgtacaagagcgagctggaagagcagctcacacctgtagctgaagaaacacgcgcacggttgtctaaagaactccaggctgctcaggcccgcttgggagcagatatggaggacgtctgtggaagactcgtccagtatcggggcgaggtgcaggccatgttgggacaaagtacggaagagcttcgggtaagattggcaagccacctcaggaaactgagaaagagactcctgagagacgcggatgacctgcagaaatgtcttgcagtgtaccaagctggagctcgcgaaggcgctgaacggggactgagtgcgattagagaacgattgggccctcttgttgaacaggggagggttagagcggcgactgtcggttctctggcagggcagcctctgcaagagcgcgctcaagcttggggtgaacgccttagagcccgaatggaagagatgggctctcggacccgagatcgacttgatgaggtgaaggagcaagtggcggaagttcgagctaagctggaggaacaggcccaacaaatccgactccaagccgaggcttttcaagcaaggctgaaaagctggtttgaacccttggtcgaagacatgcagcgccagtgggcgggattggttgaaaaagtccaagccgcggttggcacgtccgccgcccccgtgccaagtgacaatcactaa

SEQ ID NO: 24 - protein, 1036 aa, synthetic construct
  source 1..1036: note = miniXon huPAR.h28z APOE2
FLYNLTLQRATGISFAILGNFSGKFSRYHLLKFVNYISCLVSCAGNVFHLQNRNCVKFQILDEIQSYKGTNVYKIKTFSCHLQIHFVNPSATMQEVHDCVEDIILLRFNSEYLMWYILNVVLRGSLTDHTAFTPGFLYFRRGFTMVARLGSGCLSLFFGDLGTKQQEIVVSRGEDPERSGSGEGRGSLLTCGDVEENPGPRLEMALPVTALLLPLALLLHAARPQVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSDIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPEQKLISEEDLMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 25 - DNA, 2484 bp, synthetic construct
  source 1..2484: mol_type = other DNA; note = muPAR.h28z APOE2
atggccctgccagtaacggctctgctgctgccacttgctctgctcctccatgcagccaggcctgaagtgcagctggtggaaagcggcggcggcctggtgcagccgggccgcagcctgaaactgagctgcgcggcgagcggctttacctttagcaactatgcgatggcgtgggtgcgccaggcgccgaccaaaggcctggaatgggtggcgagcattagcaccggcggcggcaacacctattatcgcgatagcgtgaaaggccgctttaccattagccgcgataacgcgaaaaacaccctgtatctgcagatggatagcctgcgcagcgaagataccgcgacctattattgcgcgcgccagggcggcggctatagcgatagctttgattattggggccagggcgtgatggtgaccgtgagcagcggcggcggcggatctggaggtggtggctcaggtggcggaggctccgatgtgcagatgacccagagcccgagcaacctggcggcgagcccgggcgaaagcgtgagcattaactgcaaagcgagcaaaagcattagcaaatatctggcgtggtatcagcagaaaccgggcaaagcgaacaaactgctgatttatagcggcagcaccctgcagagcggcaccccgagccgctttagcggcagcggcagcggcaccgattttaccctgaccattcgcaacctggaaccggaagattttggcctgtattattgccagcagcataacgaatatccgctgacctttggcagcggcaccaaactggaaattaaacgcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgaccaactttagcctgctgaaacaggcgggcgatgtggaagaaaacccgggcccggactacaaagacgatgacgacaagatgaaagttttgtgggccgctttgttggtaacgttcttggcaggctgtcaggcgaaggttgaacaagcagtcgaaacggagccagagccagagctccgacagcagaccgaatggcaatctggtcaaaggtgggaacttgcgttgggccgattttgggattaccttagatgggtgcagacactttcagaacaggttcaggaggaattgcttagctcacaggtaactcaggagttgcgcgcacttatggacgagacgatgaaagaactcaaggcgtacaagagcgagctggaagagcagctcacacctgtagctgaagaaacacgcgcacggttgtctaaagaactccaggctgctcaggcccgcttgggagcagatatggaggacgtctgtggaagactcgtccagtatcggggcgaggtgcaggccatgttgggacaaagtacggaagagcttcgggtaagattggcaagccacctcaggaaactgagaaagagactcctgagagacgcggatgacctgcagaaatgtcttgcagtgtaccaagctggagctcgcgaaggcgctgaacggggactgagtgcgattagagaacgattgggccctcttgttgaacaggggagggttagagcggcgactgtcggttctctggcagggcagcctctgcaagagcgcgctcaagcttggggtgaacgccttagagcccgaatggaagagatgggctctcggacccgagatcgacttgatgaggtgaaggagcaagtggcggaagttcgagctaagctggaggaacaggcccaacaaatccgactccaagccgaggcttttcaagcaaggctgaaaagctggtttgaacccttggtcgaagacatgcagcgccagtgggcgggattggttgaaaaagtccaagccgcggttggcacgtccgccgcccccgtgccaagtgacaatcactaa

SEQ ID NO: 26 - protein, 827 aa, synthetic construct
  source 1..827: note = muPAR.h28z APOE2
MALPVTALLLPLALLLHAARPEVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPDYKDDDDKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 27 - protein, 7 aa, synthetic construct
  source 1..7: note = VHCDRI sequence
GFTFSNY

SEQ ID NO: 28 - protein, 6 aa, synthetic construct
  source 1..6: note = VHCDR2 sequence
STGGGN

SEQ ID NO: 29 - protein, 11 aa, synthetic construct
  source 1..11: note = VHCDR3 sequence
QGGGYSDSFDY

SEQ ID NO: 30 - protein, 9 aa, synthetic construct
  source 1..9: note = VHCDRI sequence
GFSLSTSGM

SEQ ID NO: 31 - protein, 5 aa, synthetic construct
  source 1..5: note = VHCDR2 sequence
WWDDD

SEQ ID NO: 32 - protein, 10 aa, synthetic construct
  source 1..10: note = VHCDR3 sequence
IGGSSGYMDY

SEQ ID NO: 33 - protein, 11 aa, synthetic construct
  source 1..11: note = VHCDRI sequence
KASKSISKYLA

SEQ ID NO: 34 - protein, 7 aa, synthetic construct
  source 1..7: note = VHCDR2 sequence
SGSTLQS

SEQ ID NO: 35 - protein, 9 aa, synthetic construct
  source 1..9: note = VHCDR3 sequence
QQHNEYPLT

SEQ ID NO: 36 - protein, 15 aa, synthetic construct
  source 1..15: note = VHCDRI sequence
RASESVDSYGNSFMH

SEQ ID NO: 37 - protein, 7 aa, synthetic construct
  source 1..7: note = VHCDR2 sequence
RASNLKS

SEQ ID NO: 38 - protein, 9 aa, synthetic construct
  source 1..9: note = VHCDR3 sequence
QQSNEDPWT

SEQ ID NO: 39 - protein, 11 aa, synthetic construct
  source 1..11: note = VHCDRI sequence
KASENVVTYVS

SEQ ID NO: 40 - protein, 7 aa, synthetic construct
  source 1..7: note = VHCDR2 sequence
GASNRYT

SEQ ID NO: 41 - protein, 9 aa, synthetic construct
  source 1..9: note = VHCDR3 sequence
GQGYSYPYT

SEQ ID NO: 42 - protein, 120 aa, synthetic construct
  source 1..120: note = VH of the uPAR binding fragment
EVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSS

SEQ ID NO: 43 - protein, 120 aa, synthetic construct
  source 1..120: note = VH of the uPAR binding fragment
QVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSS

SEQ ID NO: 44 - protein, 108 aa, synthetic construct
  source 1..108: note = VL of the uPAR binding fragment
DVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKR

SEQ ID NO: 45 - protein, 112 aa, synthetic construct
  source 1..112: note = VL of the uPAR binding fragment
DIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKR

SEQ ID NO: 46 - protein, 108 aa, synthetic construct
  source 1..108: note = VL of the uPAR binding fragment
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 47 - protein, 243 aa, synthetic construct
  source 1..243: note = uPAR binding fragment
EVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKR

SEQ ID NO: 48 - protein, 247 aa, synthetic construct
  source 1..247: note = uPAR binding fragment
QVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSDIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKR

SEQ ID NO: 49 - protein, 243 aa, synthetic construct
  source 1..243: note = uPAR binding fragment
QVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 50 - DNA, 735 bp, synthetic construct
  source 1..735: mol_type = other DNA; note = encoding sequence of uPAR binding fragment
gaagtccaactcgttgaaagcggcggtggtcttgtccagccaggcagatcactgaaactgtcatgcgccgccagtggcttcactttctccaattacgcaatggcgtgggttagacaggcccccacgaaaggcttggagtgggtcgcatcaatcagtacaggaggtggaaacacttactatcgcgatagtgttaaggggagattcacgattagccgggacaacgcgaaaaacacgttgtatctgcagatggactcacttagatccgaggacacagcgacttactactgtgcgaggcagggcggagggtatagtgatagctttgattactggggccagggcgtaatggtaactgttagttctggtggaggtggatcaggtggaggtggatctggtggaggtggatctgatgtgcagatgacacagagtccttcaaatttggccgcttcacccggagaatcagtaagtatcaactgtaaagcgtccaagtccatttcaaagtatttggcatggtatcaacagaagccgggaaaggcgaacaaactcctgatttatagcgggagtaccttgcagtccggcacgcctagtagattttcaggctccggttctgggaccgacttcactttgacgattcgcaatttggaaccagaggattttgggctgtactattgtcagcagcacaacgaatacccgttgacttttggtagtggtacaaagctggaaatcaagagagcggcc

SEQ ID NO: 51 - DNA, 741 bp, synthetic construct
  source 1..741: mol_type = other DNA; note = encoding sequence of uPAR binding fragment
caggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctgacatcgtgctgacccagagcccagctagcttggcagtgagcctgggacagagggctaccatcagctgcagagcttcagagagcgtggacagctacggaaacagcttcatgcactggtaccagcagaagccaggacagccacctaagctgctgatctaccgggctagcaacctgaagtccggaatccctgctcggtttagcggaagcggtagcggcaccgacttcaccctgacaatcaacccagtggaggccgacgatgtggcaacctactgctgtcagcagagcaacgaggacccatggaccttcggcggtggaaccaaactggagatcaagaga

SEQ ID NO: 52 - DNA, 729 bp, synthetic construct
  source 1..729: mol_type = other DNA; note = encoding sequence of uPAR binding fragment
caggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctaacatcgtgatgacccagtcccctaagagcatgagcatgagcgtgggcgagagagtgaccctgacctgcaaagcctccgagaacgtggtgacctacgtgagctggtaccagcagaagcctgagcagagccctaagctgctgatctacggcgcttccaacagatacaccggagtgcctgacagattcaccggcagcggaagcgcaaccgacttcaccttgaccatcagcagcgtgcaggctgaggacctggccgactaccactgcggccagggctacagctacccttacaccttcggtggaggcaccaagctggagatcaagcgg

SEQ ID NO: 53 - protein, 157 aa, synthetic construct
  source 1..157: note = uPAR binding fragment
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKP

SEQ ID NO: 54 - protein, 132 aa, synthetic construct
  source 1..132: note = uPAR binding fragment
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPW

SEQ ID NO: 55 - DNA, 471 bp, synthetic construct
  source 1..471: mol_type = other DNA; note = uPAR binding fragment coding sequence
atgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccc

SEQ ID NO: 56 - DNA, 396 bp, synthetic construct
  source 1..396: mol_type = other DNA; note = uPAR binding fragment coding sequence
atgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctgg

SEQ ID NO: 57 - protein, 15 aa, synthetic construct
  source 1..15: note = linker
GGGGSGGGGSGGGGS

SEQ ID NO: 58 - protein, 1368 aa, Streptococcus pyogenes [sequence omitted]

SEQ ID NO: 59 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 60 - protein, 1082 aa, Neisseria meningitidis
MAAFKPNPINYILGLDIGIASVGWAMVEIDEEENPIRLIDLGVRVFERAEVPKTGDSLAMVRRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHISGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 61 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 62 - DNA, 19 bp, synthetic construct
  source 1..19: mol_type = other DNA; note = guide sequence
cagggttctggatatctgt

SEQ ID NO: 63 - DNA, 20 bp, synthetic construct
  source 1..20: mol_type = other DNA; note = guide sequence
gggagtcaaagtcggtgaac

SEQ ID NO: 64 - DNA, 22 bp, synthetic construct
  source 1..22: mol_type = other DNA; note = primer
atcttgtgcgcatgtgaggggc

SEQ ID NO: 65 - DNA, 22 bp, synthetic construct
  source 1..22: mol_type = other DNA; note = primer
gcaagccaggactccaccaacc

(19) United States
(12) Patent Application Publication
(10) Pub. No.: US 2024/0131066 A1
(43) Pub. Date: Apr. 25, 2024

(54) SENOLYTIC CRISPR CAR T CELLS PRODUCED BY CRISPR-CAS9 GENOME EDITING

(71) Applicant: Wisconsin Alumni Research Foundation, Madison, WI (US)

(72) Inventors: Krishanu Saha, Madison, WI (US); Lauren Sarko, Middleton, WI (US)

(21) Appl. No.: 18/295,036

(22) Filed: Apr. 2, 2023

Related U.S. Application Data
(60) Provisional application No. 63/327,189, filed on Apr. 4, 2022.

(51) Int. Cl. A61K 35/17; A61K 39/00; C07K 14/005; C07K 16/28; C12N 5/00; C12N 5/0783; C12N 9/22; C12N 15/11

(52) U.S. Cl. CPC A61K 35/17; A61K 39/4611; A61K 39/4631; A61K 39/464429; C07K 14/005; C07K 16/2896; C12N 5/0056; C12N 5/0636; C12N 9/22; C12N 15/11; A61K 2239/15; A61K 2239/17; A61K 2239/21; A61K 2239/22; C07K 2317/622; C12N 2310/10

(57) ABSTRACT

Described herein are methods using CRISPR-Cas9 and DNA templates that can generate chimeric antigen receptors (CARs) on T cells to target the cell surface protein urokinase Plasminogen Activator Receptor (uPAR) on senescent cells. Also described are methods of preparing CAR T cells, their use to treat neurodegenerative disease, stroke, craniocerebral trauma and/or accident, or elderly individuals in need of treatment for aging.

### CROSS-REFERENCE TO RELATED APPLICATIONS

This application claims priority to U.S. Provisional Application 63/327,189 filed on Apr. 4, 2022, which is incorporated herein by reference in its entirety.

### STATEMENT REGARDING FEDERALLY SPONSORED RESEARCH & DEVELOPMENT

This invention was made with government support under GM119644 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Jul. 19, 2023 is named “WIS0068US2” and is 124,266 bytes in size. The Sequence Listing does not go beyond the disclosure in the application as filed.

### BACKGROUND

Senescence is a multifaceted cellular response to endogenous and exogenous stress signals that involves the induction of cell cycle arrest to eliminate unwanted cells. A fundamental feature of cell senescence is the senescence-associated secretory phenotype (SASP), which involves the secretion of tissue specific inflammatory, oxidative, and matrix-degrading factors that can attract immune cells and promote matrix rearrangement to eliminate senescent cell populations. However, in persistently damaged or aged tissues, senescent cell clearance can be compromised due to a lack immune cell recruitment, ultimately resulting in tissue dysfunction. To overcome these challenges researchers have looked to eliminate accumulated senescent cell populations that evade immune cell responses by developing antisenescent therapies also known as “senolytic” treatments. While these therapies yield promising therapeutic potential, new approaches for eliminating senescence cells are critically needed for the further understanding and prevention of tissue dysfunction in senescence associated disease pathologies.

Chimeric Antigen Receptor (CAR) T cell therapies redirect T cell specificity and effector potential functions to attack a desired target in an MHC-1 independent manner, bypassing requirements for peptide presentation. In this way, T cells can be engineered to activate against cell surface antigens for several different pathologies such as cancer, HIV, and fibrosis. Amor and colleagues (Nature, 583(7814), pp. 127-132, 2020) recently demonstrated the ability to reprogram CAR T cell effector function to target senescence associated pathologies by targeting the cell surface antigen urokinase Plasminogen Activator Receptor (uPAR). These T cells were manufactured with γ-retroviruses to target uPAR+ cells to eliminate senescent cell in vivo to reduce inflammation in lung and liver fibrosis. These genomes of these cells were not edited by CRISPR-Cas9, which provides new opportunities to increase the potency, specificity, and persistence of T cell therapies.

What is needed are alternative CAR T cell therapies, incorporating CRISPR-Cas9 genome editing, as potent senolytic agents.

### BRIEF SUMMARY

In an aspect, an DNA HDR template for a transgene comprising a chimeric antigen receptor (CAR) gene for inserting the transgene into a T cell expressed gene to generate CAR T cells having the composition:
(left HA)-(SA)-(first self-cleaving peptide polynucleotide or IRES)-(optional inducible control sequence)-(uPAR binding fragment polynucleotide)-(hinge domain polynucleotide)-(transmembrane domain polynucleotide)-(intracellular domain polynucleotide)-(second self-cleaving peptide polynucleotide or IRES)-(first secreted factor or first selection marker polynucleotide)-(optional third self-cleaving peptide polynucleotide or IRES)-(optional second secreted factor or second selection marker polynucleotide)-(polyA terminator)-(right HA);
or
(left HA)-(SA)-(first self-cleaving peptide polynucleotide or IRES) (uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional inducible control sequence)-(optional first secreted factor or first selection marker polynucleotide)-(optional third self-cleaving peptide polynucleotide or IRES)-(optional second secreted factor or second selection marker polynucleotide)-(polyA terminator)-(right HA);
or
(left HA)-(SA)-(first self-cleaving peptide polynucleotide or IRES) (uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional first secreted factor or first selection marker polynucleotide)-(optional third self-cleaving peptide polynucleotide or IRES)-(optional inducible control sequence)-(optional second secreted factor or second selection marker polynucleotide)-(polyA terminator)-(right HA);
wherein the left HA and the right HA are homology arms complementary to sequences on both sides of a cleavage site in the T cell expressed gene;
wherein SA is a splice acceptor site;
wherein the first, second and third self-cleaving peptide polynucleotide or IRES are polynucleotides encoding a first, second and third self-cleaving peptide or an internal ribosome entry site (IRES), respectively;
wherein the optional inducible control sequence is a regulatory sequence which provides control of protein expression in response to a small molecule inducer;
wherein the uPAR binding fragment polynucleotide is a polynucleotide encoding a polypeptide that specifically binds uPAR;
wherein the hinge domain polynucleotide encodes a CD28 or CD8α hinge domain;
wherein the transmembrane domain polynucleotide encodes a transmembrane domain;
wherein the intracellular domain polynucleotide encodes one or more intracellular domains;
wherein the first and second secreted factor polynucleotides are coding sequences for a neurotrophic factor, growth factor, or cytokine;
wherein the first and second selection marker polynucleotides are coding sequences for a detectable protein; and
   wherein the polyA terminator is a sequence-based element that defines the end of a transcriptional unit.

In another aspect, included are plasmids comprising the HDR template described above.

In another aspect, an ex vivo, virus-free method of site-specifically inserting a transgene containing a chimeric antigen receptor (CAR) gene into a T cell expressed gene to generate CAR T cells comprises
preparing the homology-directed repair (HDR) template described above,
introducing into a population of unmodified T cells a Cas9 ribonucleoprotein (RNP) and the HDR template to provide the CAR T cells,
   wherein the Cas9 RNP comprises a Cas9 protein and a guide RNA that directs double stranded DNA cleavage of a cleavage site in the T cell expressed gene, and
   wherein the transgene is specifically integrated into the cleavage site of the T cell expressed gene locus created by the Cas9 RNP in the cells, and
culturing the CAR T cells in xeno-free medium to provide a cultured population of CAR T cells having the transgene specifically integrated in the T cell expressed gene,
wherein, in the cultured population of CAR T cells, an endogenous promoter of the T cell expressed gene drives expression of the transgene, or wherein the transgene includes a promoter that drives expression of the transgene, and
wherein the CAR gene encodes a fusion protein comprising the translated anti-uPAR binding motif, hinge domain, transmembrane domain, and intracellular domain.

### BRIEF DESCRIPTION OF THE DRAWINGS

FIGS. 1A-F show generation, characterization, and potency of virus-free CRISPR (VFC) anti-muPAR-tNGFR T cells. (1A) Schematic of anti-muPAR-2A-tNGFR CAR construct targeting using the first encoding exon of the human TRAC gene (grey). SA: splice acceptor, T2A: self-cleaving peptide, mouse uPAR (muPAR) scFv: single chain variable fragment targeting murine uPAR, P2A: self-cleaving peptide, tNGFR: truncated nerve growth factor receptor, pA: rabbit β-globin polyA terminator. (1B) In-out PCR indicates proper on-target genomic integration of the CAR transgene in VFC-muPAR-tNGFR CAR cells. Control, untransfected donor-matched T cells. (1C) Incucyte Live-Cell Analysis system in vitro potency assay with murine fibroblasts at 5:1 and 1:1 effector:target ratio, averaged across two donors. The consistent increase in apoptotic cells after T cells were added at 0 hours indicates high potency of VFC-muPAR-tNGFR T cells. VFC-muPAR-tNGFR 5:1 (black circle) N=3; VFC-muPAR-tNGFR 1:1 (open circle) N=3; VFC-mCherry 1:1 (black square) N=3; mouse senescent fibroblast control (grey square) N=3. (1D) Summary of Inucyte Live-Cell Analysis over 48 hours. (1E) Schematic depicting in vivo mouse experiment timeline over a 28 day period. (1F) suPAR ELISA (R&D systems) assay results of blood serum collected from mice after 28 days post VFC-muPAR-tNGFR, VFC-mCherry, or no T cell infusion. Flow cytometry plots for transgene and TCR surface protein levels on the manufactured cell products. Y-axis shows mCherry levels and x-axis shows TCR levels on day 7 post-isolation. (1D) UTF, untransfected donor-matched T cells. (1E) DNA isolated from VFC-huPAR-mCh edited CAR T cells was subjected to “in-out” PCR and sequenced to evaluate TRAC locus integration. *p<0.05, one-way ANOVA. *p<0.01, one-way ANOVA.

FIGS. 2A-D show the generation, characterization, and potency of anti-huPAR-mCherry VFC-CART cells. (2A) Schematic of anti-huPAR-2A-mCherry CAR construct targeting using the first encoding exon of the human TRAC gene (grey). SA: splice acceptor, T2A: self-cleaving peptide, human uPAR (huPAR) scFv: single chain variable fragment targeting human uPAR, P2A: self-cleaving peptide, mCherry: fluorescent protein. pA: rabbit β-globin polyA terminator. (2B) Flow cytometry plots for transgene and TCR surface protein levels on the manufactured cell products. Y-axis shows mCherry levels and x-axis shows TCR levels on day 7 post-isolation. (2C) Incucyte Live-Cell Analysis system in vitro potency assay with human dermal fibroblasts (HDFa) at 5:1 and 1:1 effector:target ratio, averaged across two donors. The consistent increase in apoptotic cells after T cells were added at 0 hours indicates high potency of VFC-huPAR-mCherry T cells. VFC-huPAR-mCherry 5:1 (black circle) N=3; VFC-huPAR-mCherry 1:1 (open circle) N=3; VFC-mCherry 1:1 (black square) N=3; mouse senescent fibroblast control (grey square) N=3. (2D) Summary of Inucyte Live-Cell Analysis over 48 hours. *p<0.05, one-way ANOVA. *p<0.01, one-way ANOVA.

FIG. 3A-D show the generation, characterization, and potency of anti-muPAR-mCherry VFC-CART cells. (3A) Schematic of anti-muPAR-2A-mCherry CAR construct targeting using the first encoding exon of the human TRAC gene (grey). SA: splice acceptor, T2A: self-cleaving peptide, murine uPAR (huPAR) scFv: single chain variable fragment targeting murine uPAR, P2A: self-cleaving peptide, mCherry: fluorescent protein. pA: rabbit β-globin polyA terminator. (3B) Flow cytometry plots for transgene and TCR surface protein levels on the manufactured cell products. Y-axis shows mCherry levels and x-axis shows TCR levels on day 7 post-isolation. (3C) Incucyte Live-Cell Analysis system in vitro potency assay with murine senescent fibroblasts at 5:1 and 1:1 effector:target ratio, averaged across two donors. The consistent increase in apoptotic cells after T cells were added at 0 hours indicates high potency of VFC-muPAR-mCherry T cells. VFC-muPAR-mCherry 5:1 (black circle) N=3; VFC-muPAR-mCherry 1:1 (open circle) N=3; VFC-mCherry 1:1 (black square) N=3; mouse senescent fibroblast control (grey square) N=3. (3D) Summary of Inucyte Live-Cell Analysis over 48 hours. *p<0.05, one-way ANOVA. *p<0.01, one-way ANOVA.

FIGS. 4A-D show the generation, characterization, and potency of a fourth generation anti-muPAR-APOE2 VFC-CART cells. (4A) Schematic of anti-muPAR-2A-APOE2 CAR construct targeting using the first encoding exon of the human TRAC gene (grey). SA: splice acceptor, T2A: self-cleaving peptide, murine uPAR (huPAR) scFv: single chain variable fragment targeting murine uPAR, P2A: self-cleaving peptide, APOE2: Apolioprotein E 2 protein that forms lipoprotein particles and regulates lipid transport in both the central and peripheral nervous systems. pA: rabbit β-globin polyA terminator. (4B) In-out PCR indicates proper on-target genomic integration of the CAR transgene in VFC-muPAR-APOE2 CAR cells. Control, untransfected donor-matched T cells. (4C) Incucyte Live-Cell Analysis system in vitro potency assay with murine senescent fibroblasts at 5:1 and 1:1 effector:target ratio, averaged across two donors. The consistent increase in apoptotic cells after T cells were added at 0 hours indicates high potency of VFC-muPAR-APOE2 T cells. VFC-muPAR-APOE2 5:1 (black circle) N=3; VFC-muPAR-APOE2 1:1 (open circle) N=3; VFC-mCherry 1:1 (black square) N=3; mouse senescent fibroblast control (grey square) N=3. (4D) Summary of Inucyte Live-Cell Analysis over 48 hours. *p<0.05, one-way ANOVA. *p<0.01, one-way ANOVA.

The above-described and other features will be appreciated and understood by those skilled in the art from the following detailed description, drawings, and appended claims.

### DETAILED DESCRIPTION

The present disclosure builds on the production of anti-senescence CAR T cell therapies and adapts this technology with CRISPR/Cas9 and homology directed repair (HDR) to integrate a 4.5 kb second-generation anti-uPAR CAR transgene at the human TRAC locus. We describe uPAR CAR T cell product, e.g., a completely virus-free product, featuring precise genomic integration of our CAR and elimination of senescent cells in vitro. Of particular note, there is an increased presence of senescent cells in neurodegenerative diseases and the CAR T therapies described herein are particularly useful to treat neurodegenerative diseases such as Alzheimer's Disease, Down Syndrome, and Parkinson's Disease.

In an aspect, a DNA HDR template for a transgene comprising a chimeric antigen receptor (CAR) gene for inserting the transgene into a T cell expressed gene to generate CAR T cells having the composition:
(left HA)-(SA)-(first self-cleaving peptide polynucleotide or IRES)-(optional inducible control sequence)-(uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional first secreted factor or first selection marker polynucleotide)-(optional third self-cleaving peptide polynucleotide or IRES)-(optional second secreted factor or second selection marker polynucleotide)-(polyA terminator)-(right HA);
or
(left HA)-(SA)-(first self-cleaving peptide polynucleotide or IRES) (uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional inducible control sequence)-(optional first secreted factor or first selection marker polynucleotide)-(optional third self-cleaving peptide polynucleotide or IRES)-(optional second secreted factor or second selection marker polynucleotide)-(polyA terminator)-(right HA);
or
(left HA)-(SA)-(first self-cleaving peptide polynucleotide or IRES) (uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional first secreted factor or first selection marker polynucleotide)-(optional third self-cleaving peptide polynucleotide or IRES)-(optional inducible control sequence)-(optional second secreted factor or second selection marker polynucleotide)-(polyA terminator)-(right HA);
wherein the left HA and the right HA are homology arms complementary to sequences on both sides of a cleavage site in the T cell expressed gene;
wherein SA is a splice acceptor site;
wherein the first, second and third self-cleaving peptide polynucleotide or IRES are polynucleotides encoding a first, second and third self-cleaving peptide or an internal ribosome entry site (IRES), respectively;
wherein the optional inducible control sequence is a regulatory sequence which provides control of protein expression in response to a small molecule inducer;
wherein the uPAR binding fragment polynucleotide is a polynucleotide encoding a polypeptide that specifically binds uPAR;
wherein the hinge domain polynucleotide encodes a CD28 or CD8α hinge domain;
wherein the transmembrane domain polynucleotide encodes a transmembrane domain;
wherein the intracellular domain polynucleotide encodes one or more intracellular domain(s);
wherein the first and second secreted factor polynucleotides are coding sequences for a neurotrophic factor, growth factor, or cytokine;
wherein the first and second selection marker polynucleotides are coding sequences for a detectable protein; and
wherein the polyA terminator is a sequence-based element that defines the end of a transcriptional unit. In an aspect, the DNA HDR template is virus-free. In another aspect, the virus-free DNA HDR template is double-stranded.

### HA

As used herein, homology arms (HA) are homology arms are complementary to sequences on both sides of the cleavage site in the T cell expressed gene. The homology arms guide insertion of a synthetic DNA sequence into the T cell expressed gene by endogenous DNA repair of the double-stranded DNA cleavage induced by Cas9 RNP. The homology arms are 50 to 3000 nucleotides in length and are complementary to sequences on either side of the cut site in the T cell expressed gene to facilitate incorporation of the synthetic DNA sequence into the genome of the T cell. Small sequence variations (<100 bases) from complementary sequences could be included to enable barcoding or tracking of various cell types or to increase efficiencies of insertion of the synthetic DNA sequence.

In an aspect, the length of the homology arms influences the efficiency of synthetic DNA sequence integration. In an aspect, the homology arms are 400 to 1000 base pairs, specifically 450 to 750 base pairs long.

In an aspect, the left homology arm includes 383 to 588 bp of the TRAC locus directly upstream of the cutsite, and the right homology arm includes 391 to 499 bp of the TRAC locus directly downstream of the cutsite.

### Splice Acceptor

The splice acceptor site (SA) assists in the splicing of the synthetic DNA sequence into the transcript generated from the endogenous T cell expressed gene. The site at the 3′ end of an intron typically contains an SA. Therefore, after homology directed repair, the SA in the integrated sequence before the synthetic CAR gene assists in splicing in the CAR and downstream sequences into the endogenous transcript driven by the T cell expressed gene promoter (e.g., TRAC promoter).

### Self-Cleaving Peptides or Ires

A self-cleaving peptide sequence, e.g., T2A, assists in the separation or cleavage of the translated peptide of the protein product encoded by the synthetic DNA sequence from the protein product of the native T cell expressed gene. Exemplary self-cleaving peptides sequences include viral 2A peptides such as a porcine teschovirus-1 (P2A) peptide, a Thosea asigna virus (T2A) peptide, an equine rhinitis A virus (E2A) peptide, or a foot-and-mouth disease virus (F2A) peptide.

An internal ribosome entry site (IRES) is a site that provides initiation of translation from an internal region of the mRNA. An IRES provides co-expression of two proteins from the same mRNA.

### Inducible Control Sequence

As used herein an inducible control sequence is a regulatory sequence which takes advantage of alternative RNA splicing to provide control of protein expression in response to a small molecule inducer. An exemplary inducible control sequence is Xon which is described in Monteys et al., “Regulated control of gene therapies by drug-induced splicing”, Nature, 596, pp. 291-95 (2021). By using the Xon element upstream of our CAR sequence, transcription and subsequent translation of the uPAR binding fragment can be controlled using an oral dosing of the inducer drug treatment LMI070.

### Upar Binding Fragment

uPAR is the receptor for urokinase-type plasminogen activator (uPA), which promotes the degradation of the extracellular matrix components. uPAR expression is increased in many human cancers. As described in Amor et al., “Senolytic CAR T cells reverse senescence-associated pathologies”, Nature, 583, pp. 127-132 (2020), uPAR is induced on the surface of senescent cells. Amor also described uPAR specific CAR T cells prepared using retroviral vectors for the treatment of senescence-associated diseases. These cells drove expression of uPAR by retroviral promoters and did not modify the TRAC gene, resulting in intact TCR protein on the surface and intact signaling by the receptor.

As used herein, a uPAR binding fragment is a polynucleotide encoding a polypeptide that specifically binds uPAR. WO 2020/0160518, incorporated by reference herein for its description of uPAR binding fragments and polypeptides, describes uPAR antigen binding fragments (e.g., scFv).

In an embodiment, the uPAR binding fragment is an extracellular antigen-binding domain (e.g., human scFv) comprising a heavy chain variable (VH) region and a light chain variable (VL) region, optionally linked with a linker sequence, for example a linker peptide, between the heavy chain variable (VH) region and the light chain variable (VL) region. In certain embodiments, the extracellular antigen-binding domain is a human scFv-Fc fusion protein or full length human IgG with VH and VL regions.

In certain non-limiting embodiments, the uPAR binding fragment of the presently disclosed CAR can comprise a linker connecting the heavy chain variable (VH) region and light chain variable (VL) region of the extracellular antigen-binding domain.

In an aspect, the uPAR binding fragment comprises a VHCDRI sequence, a VHCDR2 sequence, and a VHCDR3 sequence of GFTFSNY (SEQ ID NO: 27), STGGGN (SEQ ID NO: 28), and QGGGYSDSFDY (SEQ ID NO: 29); or GFSLSTSGM (SEQ ID NO: 30), WWDDD (SEQ ID NO: 31), and IGGSSGYMDY (SEQ ID NO: 32) respectively. Additionally or alternatively, in some embodiments, the uPAR binding fragment (e.g., scFv) comprises a VLCDRI sequence, a VLCDR2 sequence, and a VLCDR3 sequence of KASKSISKYLA (SEQ ID NO: 33), SGSTLQS (SEQ ID NO: 34), and QQHNEYPLT (SEQ ID NO: 35); RASESVDSYGNSFMH (SEQ ID NO: 36), RASNLKS (SEQ ID NO: 37), and QQSNEDPWT (SEQ ID NO: 38); or KASENVVTYVS (SEQ ID NO: 39), GASNRYT (SEQ ID NO: 40), and GQGYSYPYT (SEQ ID NO: 41), respectively.

Additionally or alternatively, in some embodiments, the amino acid sequence of the VH of the uPAR binding fragment (e.g., scFv) is:

(SEQ ID NO: 42)
EVQLVESGGGLVQPGRSLKLSCAASGFTFSNYAMAWVRQA
PTKGLEWVASISTGGG NT YYRD S VKGRFTISRDNAK
NTL YLQMD SLRSEDT AT YYCARQGGGYSD SFD YW
G QGVMVTVSS,
or
(SEQ ID NO: 43)
Q VTLKE S GPGILQP SQTLSLTCSFSGESLSTS GMG
V GWIRQP S GKGLE WLAHI WWDD DKRYNPALKSRL
TISKDPSSNQVFLKIASVDTADIATYYCVRIGGSSGYMDY
WGQGT SVTVSS.

Additionally or alternatively, in some embodiments, the amino acid sequence of the VL of the uPAR binding fragment (e.g., scFv) is:

(SEQ ID NO: 44)
DVQMTQSPSNLAASPGESVSINCKASKSISKYLAWYQQKP
GKANKLLIYSGSTLQSG TPSRFSGSGS GTDFTLTIRNL
EPEDF GL YY CQ QHNE YPLTF GS GTKLEIKR,
(SEQ ID NO: 45)
DI VLT Q SP ASL AV SLGQRATI S CRASE S VD
S Y GN SFMHW YQQKPGQPPKLLI YRASNL KSGIP
ARFSGSGSGTDFTLTINPVEADDVATYCCQQSNEDPWTFG
GGTKLEIKR,
or
(SEQ ID NO: 46)
NIVMT Q SPKSMSMS VGERVTLT CKASENVVTYV SW
Y QQKPEQ SPKLLIYGASNRYT GVPDRFTGSGSATDFT
LTISSVQAEDLADYHCGQGYSYPYTFGGGTKLEIKR.

Additionally or alternatively, in some embodiments, the uPAR binding fragment (e.g., scFv) comprises an amino acid sequence selected from the group consisting of:

(SEQ ID NO: 47)
EVQLVESGGGLVQPGRSLKLSCAASGFTFSNYAMAWVRQA
PTKGLEWVASISTGGG NT YYRD S VKGRFTISRDNAK
NTL YLQMD SLRSEDT AT YYCARQGGGYSD SFD YW
G QGVMVTVSSGGGGSGGGGSGGGGSDVQMTQSPSNLAAS
PGESVSINCKASKSISKYL AWYQQKPGK ANKLLIY S
GS TLQS GTP SRF S GS GS GTDFTLTIRNLEPEDF
GL YY C QQH NE YPLTF GSGTKLEIKR;
(SEQ ID NO: 48)
Q VTLKE S GPGILQP SQTLSLTCSFSGFSLSTS GMG
V GWIRQP S GKGLE WLAHI WWDD DKRYNPALKSRL
TISKDPSSNQVFLKIASVDTADIATYYCVRIGGSSGYMDY
WGQGT SVTVSSGGGGSGGGGSGGGGSDIVLTQSPASLAV
SLGQRATISCR ASESVDSYGNSF MHWYQQKPGQPPKLL
IYRASNLKSGIPARFSGSGSGTDFTLTINPVEADDVATYC
CQ Q SNEDP WTFGGGTKLEIKR;
and
(SEQ ID NO: 49)
Q VTLKE S GPGILQP SQTLSLTCSFSGFSLSTS GMG
V GWIRQP S GKGLE WLAHI WWDD DKRYNP ALKSR
LTI SKDP S SN Q VFLKI AS VDT ADI AT YY C
VRIGGS S GYMD YWGQGT S VT V S S GGGGS GG
GGS GGGGSNI VMT QSPKSMSMS VGERVTLT CK AS
ENVVT YV S W YQQKPEQSPKLLIYGASNRYTGVPDRF
TGSGSATDFTLTISSVQAEDLADYHCGQGY S YP YTF
GGGTKLEIKR.

In an aspect, the uPAR binding fragment (e.g., scFv) is encoded by a nucleic acid sequence such as:

(SEQ ID NO: 50)
GAAGTCCAACTCGTTGAAAGCGGCGGTGGTCTTGTCCAGC
CAGGCAGATCACTG AAACTGTCATGCGCCGCCAGTGGCT
TCACTTTCTCCAATTACGCAATGGCGTGGG TT AGAC A
GGCCCCC ACGAAAGGCTTGGAGT GGGTCGC ATC AAT
CAGT AC AGGAG GT GGAAAC ACTT ACT ATCGCGA
T AGT GTTAAGGGGAGATTC ACGATTAGCCGGG AC A
ACGCGAAAAAC ACGTTGTATCTGC AGATGGACTC ACT
T AGATCCGAGGAC A C AGCGACTT ACT ACTGTGCG
AGGC AGGGCGGAGGGT AT AGT GAT AGCTTT GATT
ACTGGGGCCAGGGCGTAATGGTAACTGTTAGTTCTGGTGG
AGGTGGATCAGGTG GAGGTGGATCTGGTGGAGGTGGATC
TGATGTGCAGATGACACAGAGTCCTTCAAATTTGGCCGCT
TCACCCGGAGAATCAGTAAGTATCAACTGTAAAGCGTCCA
AGTCC ATTTC AAAGT ATTTGGC ATGGTAT C AACA
GAAGCCGGGAAAGGCGAAC AAACTCCTGATTTATAGCGG
GAGTACCTTGCAGTCCGGCACGCCTAGTAGATTTTCAGGC
TCCGGTTCTGGGACCGACTTCACTTTGACGATTCGCAATT
TGGAACCAGAGGATTTTGGGCTGTACTATTGTCAGCAGCA
CAACGAATACCCGTTGACTTTTGGTAGTGGTACAAAGCTG
GAAATCAAGAGAGCGGCC;
(SEQ ID NO: 51)
CAGGTGACCCTGAAGGAGTCCGGCCCCGGCATCCTGCAGC
CCAGCCAGACCCTGAGCCTGACCTGCTCCTTCAGCGGCTT
CTCCCTGTCCACCTCCGGCATGGGCGTGGGCTGGATCAGA
CAGCCCAGCGGCAAGGGCCTGGAGTGGCTGGCCCACATCT
GGT GGGACGATGACAAGAGATACAACCCCGCTCTGAAGA
GCCGGCTGACAATCAGCAAGGACCCTAGCAGTAACCAGGT
GTTCCTGAAGATCGCTTCCGTGGACACAGCAGACATCGCA
ACATACTATTGCGTGCGGATCGGCGGAAGCAGTGGATACA
TGGACTACTGGGGAC AGGGAACC AGCGTGACCGT GAG
CAGT GGT GGAGGT GGAT CAGGTGGAGGTGGATCTGG
TGGAGGTGGATCTGACATCGTGCTGACCCAGAGCCCAGCT
AGCTTGGCAGTGAGCCTGGGACAGAGGGCTACCATCAGCT
GCAGAGCTTCAGAGAGCGTGGACAGCTACGGAAACAGCTT
CATGCACTGGTACCAGCAGAAGCCAGGACAGCC ACCT A
AGCT GCTGATCT ACCGGGCT AGC AACCT GAAGTCC
GGAATCCCTGCTCGGTTTAGCGGAAGCGGTAGCGGCACCG
ACTTCACCCTGACAATCAACCCAGTGGAGGCCGACGATGT
GGC AACCT ACTGCTGT C AGC AGAGC AACGAGGA
CCC AT GGACCTTCGGCGGT GGAACC AAACT GGAGA
T CAAGAGA;
and
(SEQ ID NO: 52)
CAGGTGACCCTGAAGGAGTCCGGCCCCGGCATCCTGCAGC
CCAGCCAGACCCTGAGCCTGACCTGCTCCTTCAGCGGCTT
CTCCCTGTCCACCTCCGGCATGGGCGTGGGCTGGATCAGA
CAGCCCAGCGGCAAGGGCCTGGAGTGGCTGGCCCACATCT
GGTGGGACGATGACAAGAGATACAACCCCGCTCTGAAGAG
CCGGCTGACAATCAGCAAGGACCCTAGCAGTAACCAGGTG
TTCCTGAAGATCGCTTCCGTGGACACAGCAGACATCGCAA
CATACTATTGCGTGCGGATCGGCGGAAGCAGTGGATACAT
GGACTACTGGGGACAGGGAACCAGCGTGACCGTGAGCAGT
GGTGGAGGTGGATCAGGTGGAGGTGGATCTGGTGGAGGTG
GATCTAACATCGTGATGACCCAGTCCCCTAAGAGCATGAG
CATGAGCGTGGGCGAGAGAGTGACCCTGACCTGCAAAGCC
TCCGAGAACGTGGTGACCTACGTGAGCTGGTACCAGCAGA
AGCCTGAGCAGAGCCCTAAGCTGCTGATCTACGGCGCTTC
CAACAGATACACCGGAGTGCCTGACAGATTCACCGGCAGC
GGAAGCGCAACCGACTTCACCTTGACCATCAGCAGCGTGC
AGGCTGAGGACCTGGCCGACTACCACTGCGGCCAGGGCTA
CAGCTACCCTTACACCTTCGGTGGAGGCACCAAGCTGGAG
ATCAAGCGG.

Additionally or alternatively, in some embodiments, the uPAR binding fragment (e.g., scFv) is encoded by a nucleic acid sequence that has at least 80%, 85%, 90%, 95%, 96%, 97%, 98%, or 99% sequence identity to any one of SEQ ID NOs: 36-38. In some embodiments, the uPAR binding fragment (e.g., scFv) is encoded by a nucleic acid that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 50-52.

In some embodiments, the chimeric antigen receptor comprises a uPAR binding fragment (e.g., a uPA fragment) comprising the amino acid sequence:

(SEQ ID NO: 53)
MRALL ARLLLC VLVV SD SKGSNELHQ VP SN CDC
LN GGT C V SNKYFSNIHW CN CPKKFGGQHCEIDKS
KTCYEGNGHFYRGKASTDTMGRPCLPWNSATVLQQTYHAH
RSDA LQLGLGKHNY CRNPDNRRRP W C YV Q V GL
KPL V QECMVHDCADGKKP;
or
(SEQ ID NO: 54)
MRALL ARLLLC VLVV SD SKGSNELHQ VP SN CDC
LN GGT C V SNKYFSNIHW CN CPKKFGGQHCEIDKS
KTCYEGNGHFYRGKASTDTMGRPCLPWNSATVLQQTYHAH
RSDA LQLGLGKHNY CRNPDNRRRP W.

Additionally or alternatively, in some embodiments, the uPAR binding fragment (e.g., uPa fragment) comprises an amino acid sequence that has at least 80%, 85%, 90%, 95%, 96%, 97%, 98%, or 99% sequence identity to SEQ ID NO: 53 or SEQ ID NO: 54. In some embodiments, the uPAR binding fragment (e.g., uPa fragment) comprises an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 53 or SEQ ID NO: 54.

Additionally or alternatively, in some embodiments, the uPAR binding fragment (e.g., a uPAR fragment) is encoded by a nucleic acid sequence:

(SEQ ID NO: 55)
ATGAGAGCCCTGCTGGCGCGCCTGCTTCTCTGCGTCCTGG
TCGTGAGCGACTCCA AAGGC AGC AAT GAACTTC AT
C AAGTTCC ATCGAACTGT GACTGTCTAAATGGAGGAA
CATGTGTGTCCAACAAGTACTTCTCCAACATTCACTGGTG
CAACTGCCCAAA GAAATTCGGAGGGC AGC ACTGT GA
AAT AG AT AAGTCAAAAACCTGCT ATGAGGGGAATGG
TCACTTTTACCGAGGAAAGGCCAGCACTGACACCATGGGC
CGGCCCTGCCTGCCCTGGAACTCTGCCACTGTCCTTCAGC
AAACGTACCATGCCCACAGATCT GAT GCTCTTC AGCT
GGGCCTGGGGAAAC AT AATT ACTGC AGGAACCC AG
AC AAC CGGAGGCGACCCTGGTGCTAT GT GC AGGT
GGGCCT AAAGCCGCTTGTCC AAGAG T GC AT GGT
GC ATGACTGCGC AGAT GGAAAAAAGCCC;
or
(SEQ ID NO: 56)
ATGAGAGCCCTGCTGGCGCGCCTGCTTCTCTGCGTCCTGG
TCGTGAGCGACTCCA AAGGC AGC AAT GAACTTC AT
C AAGTTCC ATCGAACTGT GACTGTCTAAATGGAGGAA
CATGTGTGTCCAACAAGTACTTCTCCAACATTCACTGGTG
CAACTGCCCAAA GAAATTCGGAGGGC AGC ACTGT GA
AAT AGAT AAGTCAAAAACCTGCT AT GAGGGGAATGG
TCACTTTTACCGAGGAAAGGCCAGCACTGACACCATGGGC
CGGCCCTGCCTGCCCTGGAACTCTGCCACTGTCCTTCAGC
AAACGTACCATGCCCACAGATCT GAT GCTCTTC AGCT
GGGCCTGGGGAAAC AT AATT ACTGC AGGAACCC A
GAC AAC CGGAGGCGACCCTGG

Additionally or alternatively, in some embodiments, the uPAR binding fragment is encoded by a nucleic acid sequence that has at least 80%, 85%, 90%, 95%, 96%, 97%, 98%, or 99% sequence identity to any one of SEQ ID NOs: 55 or 56. In some embodiments, the uPAR binding fragment is encoded by a nucleic acid that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 55 or 56.

In an aspect, the uPAR binding fragment is an antibody fragment. As used herein, the term “single-chain variable fragment” ˜ or “scFv” is a fusion protein of the variable regions of the heavy (VH) and light chains (VL) of an immunoglobulin (e.g., mouse or human) covalently linked to form a VH:VL heterodimer. The heavy (VH) and light chains (VL) are either joined directly or joined by a peptide-encoded linker (e.g., about 10, 15, 20, 25 amino acids), which connects the N-terminus of the VH with the C-terminus of the VL, or the C-terminus of the VH with the N-terminus of the VL. The linker is may be rich in glycine for flexibility, as well as serine or threonine for solubility. The linker can link the heavy chain variable region and the light chain variable region of the extracellular antigen binding domain. In certain embodiments, the linker comprises amino acids having the sequence GGGGS GGGGS GGGGS (SEQ ID NO: 57).

A specific uPAR binding fragment includes a heavy chain variable fragment and a light chain variable fragment, optionally connected by a linker (SEQ IDs 40-41).

### Car Domains

Typically, the antigen-specific extracellular domain (uPAR binding fragment) is linked to the intracellular domain of the CAR by a transmembrane domain, e.g., derived from a CD4, CD8α, CD28, IgG and/or or CD3zeta transmembrane domain. The transmembrane domain traverses the cell membrane, anchors the CAR to the T cell surface, and connects the extracellular domain to the intracellular signaling domain, thus impacting expression of the CAR on the T cell surface. The uPAR binding fragment is linked to the intracellular domain by a hinge domain such as a CD28 or CD8α hinge domain. The hinge domain provides flexibility to the uPAR binding fragment and improves efficacy. CARs may also further comprise one or more costimulatory domain and/or one or more spacer. A costimulatory domain is derived from the intracellular signaling domains of costimulatory proteins that enhance cytokine production, proliferation, cytotoxicity, and/or persistence in vivo. A spacer or hinge connects (i) the antigen-specific extracellular domain to the transmembrane domain, (ii) the transmembrane domain to a costimulatory domain, (Hi) a costimulatory domain to the intracellular domain, and/or (iv) the transmembrane domain to the intracellular domain. For example, inclusion of a spacer domain (e.g., IgG1, IgG2, IgG4, CD28, CD8) between the antigen-specific extracellular domain and the transmembrane domain may affect flexibility of the antigen-binding domain and thereby CAR function. Transmembrane domains, costimulatory domains, and spacers are known in the art. Exemplary costimulatory domains include OX40, 41BB, ICOS, CD27, CD40, CD40L or a TLR.

### Secreted Factors

The first and second secreted factors are a coding sequence for a neurotrophic factor or cytokine. Secreted factors can include neuroprotective, pro-regenerative secreted factors such as APOE2, sAPPa; pro-memory secreted factors such as IL-4, IL-10; growth factors like BDNF, NGF; factors that attract pro-regenerative immune cells such as IL-1, IL-6, TNF-alpha, IFN-gamma; and the like. Exemplary secreted factors include a pro-regenerative secreted factor, a pro-memory secreted factor, growth factor, or a factor that attracts pro-regenerative immune cells SELECTION MARKERS

In an aspect, the synthetic DNA sequence comprises a coding sequence for a selection marker which can be selectable cell surface receptors such as truncated NGFR (tNGFR) or a fluorescent protein such as mCherry, mKate, GFP, BFP, RFP, CFP, YFP, mCyan, mOrange, tdTomato, mBanana, mPlum, mRaspberry, mStrawberry, and mTangerine.

### Polya Terminator

The polyadenylation (polyA) terminator is a sequence-based element that defines the end of a transcriptional unit within the synthetic DNA sequence and initiate the process of releasing the newly synthesized RNA from the transcription machinery. Exemplary polyA terminators are rabbit beta-globin polyA and a bovine growth hormone polyA.

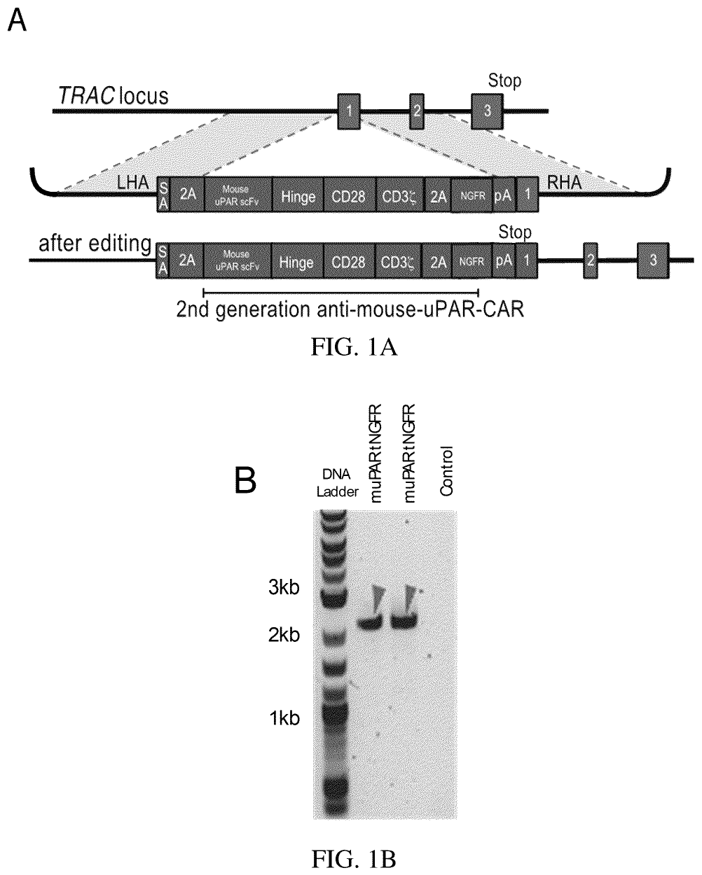

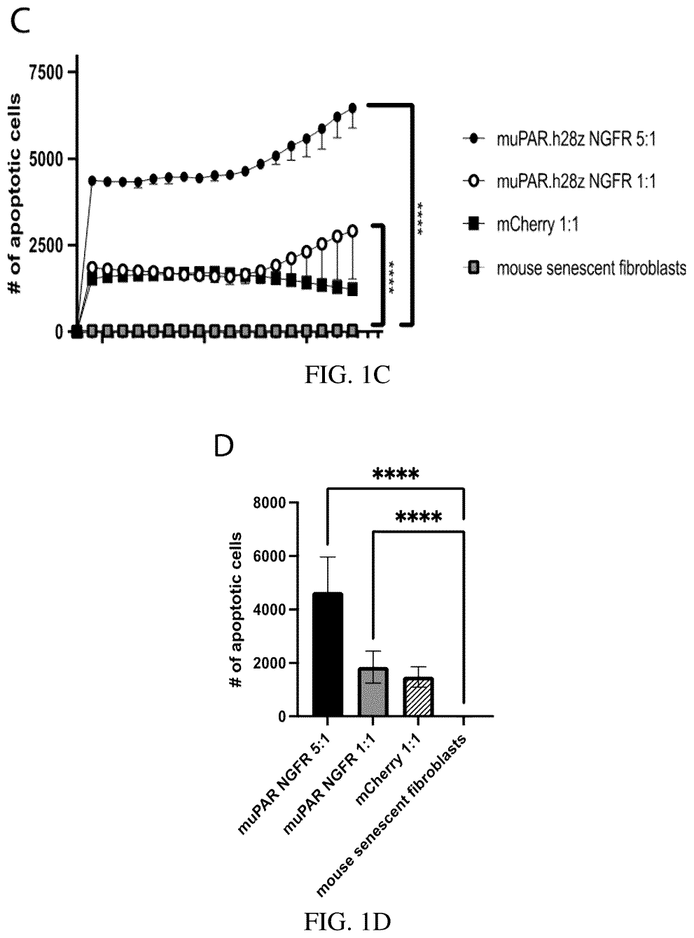

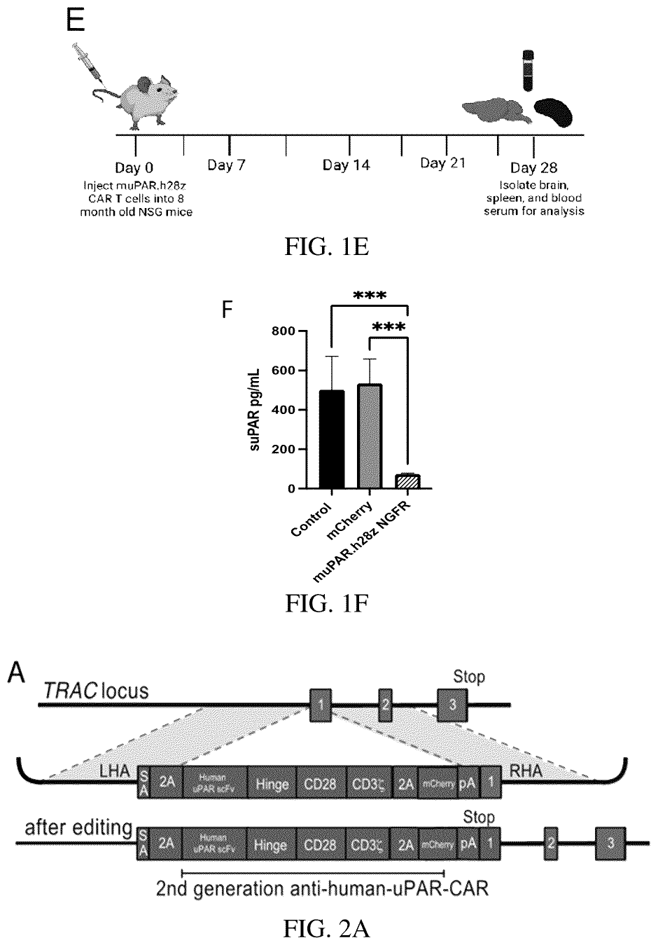

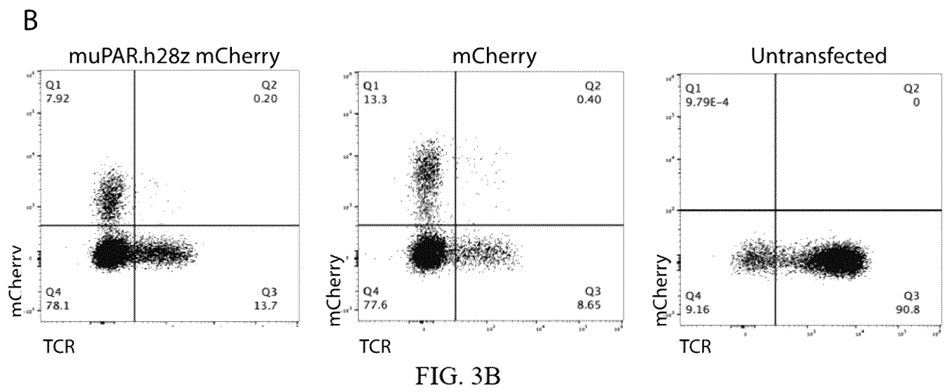

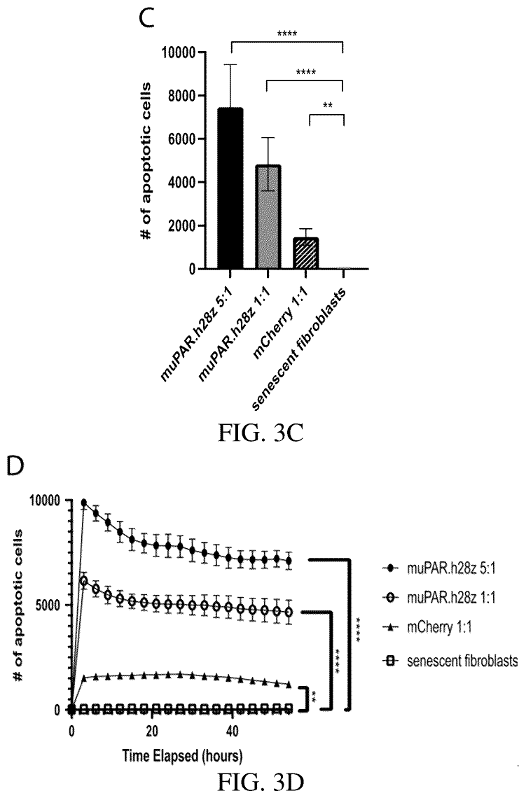

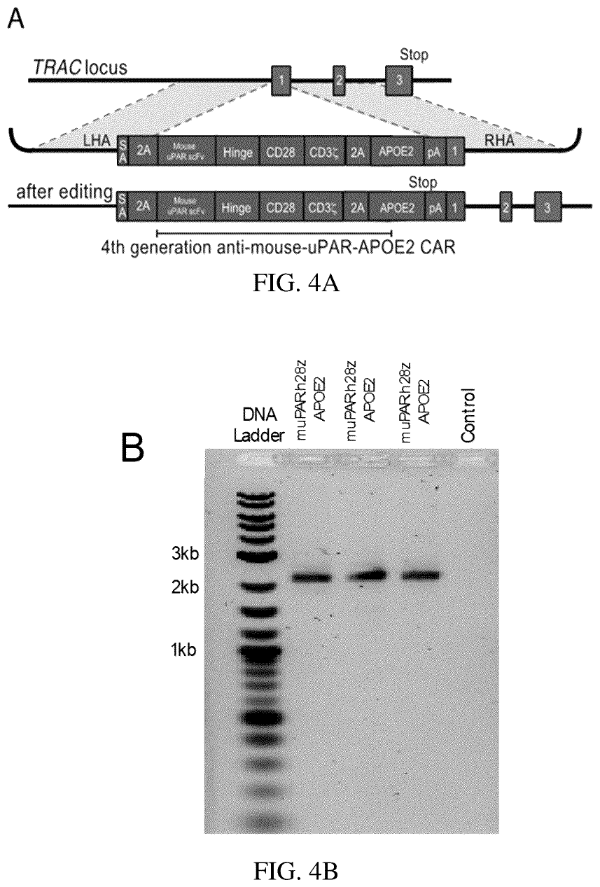

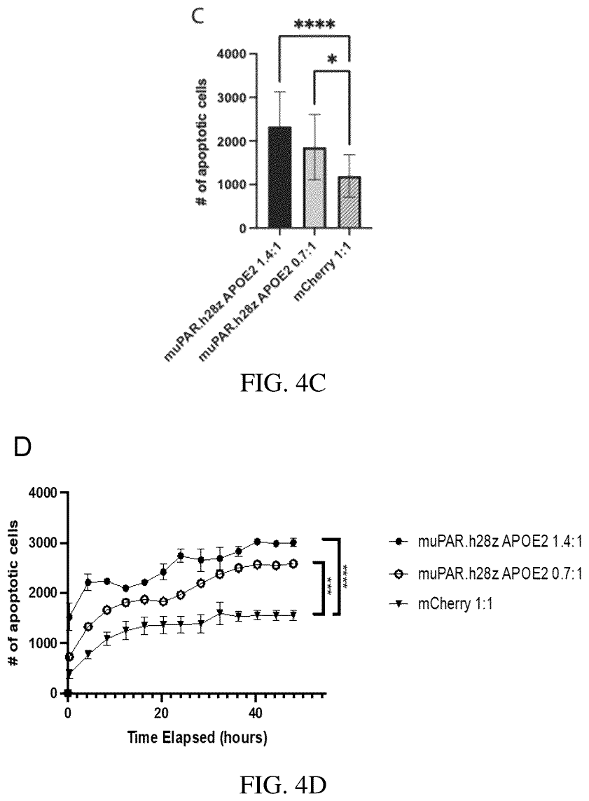

FIG. 1A is a schematic of an anti-huPAR-CAR-2A-mCh targeting strategy. FIG. 3 is a schematic of an anti-muPAR-CAR-2A-NGFR targeting strategy. muPAR is the scFV VH and VL of mouse anti-uPAR binding fragment with a CD8 linker. The CD28 hinge and transmembrane domain were used. The zeta chain is a CD3 zeta chain. NGFR is a truncated nerve growth factor affinity receptor. mCh is the mCherry fluorescent protein.

Exemplary sequences of the present disclosure include the following:
SEQ ID NO: 1—DNA: muPAR.h28z tNGFR
SEQ ID NO: 2—protein: muPAR.h28z tNGFR
SEQ ID NO: 3—DNA: huPAR.h28z tNGFR
SEQ ID NO: 4—protein: huPAR.h28z tNGFR
SEQ ID NO: 5 DNA: huPAR.h28z mCherry
SEQ ID NO: 6 protein: huPAR.h28z mCherry
SEQ ID NO: 7 DNA: muPAR.m28z tNGFR
SEQ ID NO: 8 protein: muPAR.m28z tNGFR
SEQ ID NO: 9 DNA: muPAR.h28z mCherry
SEQ ID NO: 10 protein: muPAR.h28z mCherry
SEQ ID NO: 11 DNA: muPAR.m28z mCherry
SEQ ID NO: 12 protein: muPAR.m28z mCherry
SEQ ID NO: 13 DNA: muPAR.m28z APOE2
SEQ ID NO: 14 protein: muPAR.m28z APOE2
SEQ ID NO: 15 DNA: huPAR.h28z APOE2
SEQ ID NO: 16 protein: huPAR.h28z APOE2
SEQ ID NO: 17 DNA: miniXon huPAR.h28z mCherry
SEQ ID NO: 18 protein: miniXon huPAR.h28z mCherry
SEQ ID NO: 19 DNA: miniXon muPAR.m28z mCherry
SEQ ID NO: 20 protein: miniXon muPAR.m28z mCherry
SEQ ID NO. 21 DNA: miniXon muPAR.m28z APOE2
SEQ ID NO. 22 protein: miniXon muPAR.m28z APOE2
SEQ ID NO: 23 DNA: miniXon huPAR.h28z APOE2
SEQ ID NO: 24 protein: miniXon huPAR.h28z APOE2
SEQ ID NO: 25 DNA: muPAR.h28z APOE2
SEQ ID NO: 26 protein: muPAR.h28z APOE2

### Plasmids

Also included herein is a plasmid comprising the virus-free double-stranded HDR template described herein. Exemplary plasmids are non-viral expression vectors such as pUC57 and pUC57-Mini.

### Genomic Integration of the Car Expressing the Upar Binding Fragment Polynucleotide

In a gene editing method, guide RNAs direct Cas9 nuclease to create a double stranded DNA break at the target locus. DNA repair involving the DNA template containing the synthetic CAR sequence then allows the integration of the CAR described herein into T cells to provide genome-edited T-cells.

In an aspect, an ex vivo, virus-free method of site-specifically inserting a transgene containing a chimeric antigen receptor (CAR) gene into a T cell expressed gene to generate CAR T cells comprises
preparing the virus-free homology-directed repair (HDR) template described above,
introducing into a population of unmodified T cells a Cas9 ribonucleoprotein (RNP) and the HDR template to provide the CAR T cells,
   wherein the Cas9 RNP comprises a Cas9 protein and a guide RNA that directs double stranded DNA cleavage of a cleavage site in the T cell expressed gene, and
   wherein the transgene is specifically integrated into the cleavage site of the T cell expressed gene locus created by the Cas9 RNP in the cells, and
culturing the CAR T cells in xeno-free medium to provide a cultured population of CAR T cells having the transgene specifically integrated in the T cell expressed gene,
wherein, in the cultured population of CAR T cells, an endogenous promoter of the T cell expressed gene drives expression of the transgene, or wherein the transgene includes a promoter that drives expression of the transgene, and
wherein the CAR gene encodes a fusion protein comprising the translated anti-uPAR binding motif, hinge, transmembrane domain, and intracellular domain.

As used herein, “introducing” means refers to the translocation of the Cas9 ribonucleoprotein and a DNA template from outside a cell to inside the cell, such as inside the nucleus of the cell. Introducing can include transfection, electroporation, contact with nanowires or nanotubes, receptor mediated internalization, translocation via cell penetrating peptides, liposome mediated translocation, transduction with putative non-integrating viruses (e.g., adeno-associated virus, AAV), viral-like particles (VLPs), and the like.

Unmodified T cells include autologous T cells that are collected from a patient, such as a cancer patient, by peripheral blood draw or leukapheresis. Unmodified T cells can also include T cells from allogeneic healthy donors or induced pluripotent stem cells which can be used to produce universal T cells for administration to a patient. T cells are generally modified ex vivo, that is outside of the patient, and then the modified T cells such as CAR T cells are returned to the patient, such as by intravenous infusion, subcutaneous, intratumoral, intraperitoneal or intravenous or intracerebroventricular infusion or intracerebral injection.

Genome editing of the T cells as described herein uses a CRISPR system, or Cas9 ribonucleoprotein. CRISPR refers to the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner. In type II systems, guide RNA interacts with Cas9 and directs the nuclease activity of Cas9 to target DNA sequences complementary to those present in the guide RNA. Guide RNA base pairs with complementary sequences in target DNA. Cas9 nuclease activity then generates a double-stranded break in the target DNA.

CRISPR/Cas9 is a ribonucleoprotein (RNP) complex. CRISPR RNA (crRNA) includes a 20 base protospacer element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

Guide RNA, or gRNA, can be in the form of a crRNA/tracrRNA two guide system, or an sgRNA single guide RNA. The guide RNA is capable of directing Cas9-mediated cleavage of target DNA. A guide RNA thus contains the sequences necessary for Cas9 binding and nuclease activity and a target sequence complementary to a target DNA of interest (protospacer sequence).

As used herein, a guide RNA protospacer sequence refers to the nucleotide sequence of a guide RNA that binds to a target genomic DNA sequence and directs Cas9 nuclease activity to a target DNA locus in the genome of the T cell such the TRAC gene, a T cell receptor beta subunit constant gene (TRBC), AAVS1 (i.e., PPP1R12C), TET2, FAS, BID, CTLA4, PDCD1, CBLB, PTPN6, CIITA and B2M genes. In some embodiments, the guide RNA protospacer sequence is complementary to the target DNA sequence. “Complementary” or “complementarity” refers to specific base pairing between nucleotides or nucleic acids. Base pairing between a guide RNA and a target region in exon 1 of the TRAC gene can be via a DNA targeting sequence that is perfectly complementary or substantially complementary to the guide RNA. As described herein, the protospacer sequence of a single guide RNA may be customized, allowing the targeting of Cas9 activity to a target DNA of interest.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the targeted sequence includes a protospacer adjacent motif (PAM) adjacent to the protospacer region which is a sequence recognized by the CRISPR RNP as a cutting site. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs. For example, Cas9 from Streptococcus pyogenes has a NGG trinucleotide PAM motif; the PAM motif of N. meningitidis Cas9 is NNNNGATT; the PAM motif of S. thermophilus Cas9 is NNAGAAW; and the PAM motif of T. denticola Cas9 is NAAAAC.

A “Cas9” polypeptide is a polypeptide that functions as a nuclease when complexed to a guide RNA, e.g., an sgRNA or modified sgRNA. That is, Cas9 is an RNA-mediated nuclease. The Cas9 (CRISPR-associated 9, also known as Csnl) family of polypeptides, for example, when bound to a crRNA:tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case1, Cse 2, Csy 1, Csy 2, Csy 3, GSU0054, Cas 10, Csm 2, Cmr 5, Cas10, Csx11, Csx10, and Csf 1. Additional Class 2 Cas9 polypeptides include Csn 2, Cas4, C2c1, C2c3 and Cas13a.

Exemplary Cas9 polypeptides include Cas9 polypeptide derived from Streptococcus pyogenes, e.g., a polypeptide having the sequence of the Swiss-Prot accession Q99ZW2 (SEQ ID NO: 58); Cas9 polypeptide derived from Streptococcus thermophilus, e.g., a polypeptide having the sequence of the Swiss-Prot accession G3ECR1 (SEQ ID NO: 59); a Cas9 polypeptide derived from a bacterial species within the genus Streptococcus; a Cas9 polypeptide derived from a bacterial species in the genus Neisseria meningitidis (e.g., GenBank accession number YP_003082577; WP_015815286.1 (SEQ ID NO: 60)); a Cas9 polypeptide derived from a bacterial species within the genus Treponema denticola (e.g., GenBank accession number EMB41078 (SEQ ID NO: 61)); and a polypeptide with Cas9 activity derived from a bacterial or archaeal species. Methods of identifying a Cas9 protein are known in the art. For example, a putative Cas9 protein may be complexed with crRNA and tracrRNA or sgRNA and incubated with DNA bearing a target DNA sequence and a PAM motif.

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some embodiments, a Cas9 nuclease has an inactive (e.g., an inactivated) DNA cleavage domain, that is, the Cas9 is a nickase. Other embodiments of Cas9, both DNA cleavage domains are inactivated. This is referred to as catalytically-inactive Cas9, dead Cas9, or dCas9.

Functional Cas9 mutants are described, for example, in US20170081650 and US20170152508, incorporated herein by reference for its disclosure of Cas9 mutants.

As used herein, the term editing refers to a change in the sequence of the genome at a targeted genomic location. Editing can include inducing either a double stranded break or a pair of single stranded breaks in the genome, such as in a T cell expressed gene. Editing can also include inserting a synthetic DNA sequence into the genome of the T cell at the site of the break(s).

As used herein, a Cas9 RNP that targets a T cell expressed gene comprises a Cas9 protein and a guide RNA that directs double stranded DNA cleavage of the T cell expressed gene. The guide RNA thus includes a crRNA comprising a single-stranded protospacer sequence and a first complementary strand of a binding region for the Cas9 polypeptide, and a tracrRNA comprising a second complementary strand of the binding region for the Cas9 polypeptide, wherein the crRNA and the tracrRNA hybridize through the first and second complementary strands of the binding region for the Cas9 polypeptide. The single-stranded protospacer region of the guide RNA hybridizes to a sequence in the T cell expressed gene, directing cleavage of the T-cell expressed gene to a specific locus of the T cell expressed gene.

Exemplary T cell expressed genes which can be cleaved by the methods described herein include the AAVS1 (i.e., PPP1R12C), TET2, FAS, BID, CTLA4, PDCD1, CBLB, PTPN6, CIITA, B2M, TRAC and TRBC genes, specifically TRAC. The T cell expressed gene-targeted by Cas9 ribonucleoprotein may result in a reduction or elimination of expression of functional TRAC gene product (e.g., knockout of expression of functional TRAC gene product).

In an aspect, the T cell expressed gene is TRAC and wherein the guide RNA targets the 5′ end of the first exon of TRAC. An exemplary guide RNA useful to target the first encoding exon of TRAC comprises SEQ ID NO: 62; CAGGGTTCTGGATATCTGT or SEQ ID NO: 63; GGGAGTCAAAGTCGGTGAAC

In addition to the Cas9 RNP, the virus-free double-stranded HDR template comprising the synthetic DNA sequence is introduced into the T cells.

The genome-edited T cells are then cultured in in xeno-free medium to provide a cultured population of T cells having the synthetic DNA sequence specifically integrated in the T-cell expressed gene locus. The term “xeno” comes from the Greek “xenos” meaning strange. Xeno-free (or xenogeneic-free) therefore means free from “strange” components, or components from a “strange” species (strange being relative to the native species you're working with). In terms of cell culture, this would mean human cell lines can be cultured using human-derived components (like human serum), and it is considered xeno-free, since there is no difference between species.

As used herein culturing the genome-edited T cells in xeno-free medium can include recovery from integration of the synthetic DNA sequence and/or expansion of the edited T cell population.

In an aspect, the CAR T cells produced by the methods described herein have activity against a neurodegenerative disease, stroke, craniocerebral trauma and/or accident, or an elderly patient in need of treatment for aging, for example. Thus, the methods further comprise administering the cultured population of CAR T cells to a patient in need of treatment for a neurodegenerative disease, stroke, craniocerebral trauma and/or accident, or an elderly patient in need of treatment for aging. Exemplary neurodegenerative diseases include Alzheimer's disease, dementia, Parkinson's disease, Lewy body disease, ataxia, Huntington's disease, amyotrophic lateral sclerosis, Down syndrome, and spinal muscular atrophy.

In an aspect, administering the CAR T cells is by intravenous or intracerebroventricular infusion of intracerebral injection.

The invention is further illustrated by the following non-limiting examples.

### EXAMPLES

### Methods

Cell lines: Primary Human Dermal Fibroblasts adult (HDFa) were purchased from ATCC and maintained in Dulbecco's Modified Eagle Medium high glucose (Gibco) supplemented with 10% Fetal Bovine Serum (Gibco) and 1% penicillin-streptomycin. For drug-induced senescence experiments, trametinib (S2673) and palbociclib (S1116) were purchased from Selleck Chemicals and dissolved in DMSO to yield 10 mM stock solutions, which were stored at −80° C. Cells were treated with MEK inhibitor (25 nM) and CDK4/6 inhibitor (500 nM). The cells were induced for 48 hours, the growth medium was then changed every two days. Cortical Glutamatergic GFP+ Neurons were purchased from BrainXell. These cells were maintained in 50% Dulbecco's Modified Eagle Medium Nutrient Fixture F-12 (Gibco) and 50% Neurobasal Medium (Gibco) supplemented with 2% B27 Supplement (Thermofischer), 1% N2 Supplement (Thermofischer), 0.5 mM Glutamax™ (Gibco), BDNF 10 ng/mL (Peprotech), 10 ng/mL GDNF (Peprotech), 1 ng/mL TGF-β1 (Peprotech), Geltrex® 15 μg/mL (Thermofischer), Neuron Seeding Supplement Day 1 1× (BrainXell), Supplement K 1× (BrainXell). For drug-induced senescence experiments 300 μM Hydrogen Peroxide (Sigma Aldrich) was added to the neuron cultures for 2 hours to induce oxidative stress. After incubation, media was taken off of the cells and replaced with normal glutamatergic neuron culture. Cell lines were maintained in culture at 37° C. in 5% CO2 and tested negative for mycoplasma.

Isolation of primary T cells from healthy donors: This study was approved by the Institutional Review Board of the University of Wisconsin-Madison (#2018-0103), and informed consent was obtained from all donors. Peripheral blood was drawn from healthy donors into sterile syringes containing heparin and transferred to sterile 50 mL conical tubes. Primary human T cells were isolated using RosetteSep™ Human T Cell Enrichment Cocktail (STEMCELL Technologies). T cells were counted using a Countess™ II FL Automated Cell Counter (Thermo Fisher Scientific) with 0.4% Trypan Blue viability stain (Thermo Fisher Scientific) at a 1:1 dilution. T cells were cultured at a final density of 1 million cells/mL in ImmunoCult™—XF T cell Expansion Medium (STEMCELL) supplemented with 200 U/mL IL-2 (Peprotech) and stimulated with ImmunoCult™ Human CD3/CD28/CD2 T cell Activator (STEMCELL) immediately after isolation, per the manufacturer's instructions.

T cell culture: T cells were cultured in ImmunoCult™—XF T cell Expansion Medium at a density of 1 million cells/mL and stimulated with ImmunoCult™ Human CD3/CD28/CD2 T cell Activator (STEMCELL) for 48 hours prior to electroporation. After 24 hours post-electroporation, VFC T cells were transferred without centrifugation to 1 mL of fresh culture medium with 500 U/mL IL-2. T cells were passaged, counted, and adjusted to 1 million/mL in fresh medium+IL-2 on days 5, 7, 9, 11, and 14 after isolation.

Double-stranded DNA HDR template production: Plasmids were generated by Genscript by inserting CAR constructs into a pUC57 vector. VFC-huPAR.28z-2A-mCherry (also termed VFC-huPAR-mCh) and VFC-mCherry (also termed VFC-mCh) plasmids were transformed in 5-alpha competent E. coli (NEB) and purified using the PureYield™ MiniPrep system (Promega). PCR amplicons were generated from plasmid templates using Q5® Hot Start Polymerase (NEB) and pooled into 600 μl reactions for Solid Phase Reversible Immobilization (SPRI) cleanup (6×) using AMPure XP beads according to the manufacturer's instructions (Beckman Coulter). Each of the 600 μl starting products was eluted into 30 μl of water. Bead incubation and separation times were increased to 5 minutes, and elution time was increased to 15 minutes at 37° C. to improve overall yield. PCR products from round 1 cleanup were pooled and subjected to an ethanol precipitation to increase total concentration. Template concentration and purity was quantified using a IMPLEN NanoPhotemeter® N50. Concentrated template products were diluted in UltraPure H20 at a concentration of 2.5 μg/μl according to Nanodrop™ measurements.

SpCas9 RNP preparation: RNPs were produced by complexing a two-component gRNA to SpCas9. In brief, tracrRNA and crRNA were ordered from IDT, suspended in nuclease-free duplex buffer at 100 μM, and stored in single-use aliquots at −80° C. tracrRNA and crRNA were thawed, and 4.15 μl of each component was mixed 1:1 by volume and annealed by incubation at 37° C. for 30 minutes to form a 50 μM gRNA solution in individual aliquots for each electroporation replicate. Recombinant sNLS-SpCas9-sNLS Cas9 (Aldevron, 10 mg/ml, total 3.33 μl) was added to the complexed gRNA at a 1.2:1 molar ratio and incubated for 15 minutes at 37° C. to form an RNP. Individual aliquots of RNPs were incubated for at least 30 seconds at room temperature with HDR templates for each sample prior to electroporation.

T cell nucleofection: Following guidance from the protocols in the art, RNPs and HDR templates were electroporated 2 days after T cell isolation and stimulation. During crRNA and tracrRNA incubation, T cells were centrifuged for 3 minutes at 200 g and counted using a Countess™ II FL Automated Cell Counter with 0.4% Trypan Blue viability stain (Thermo Fisher). 4.13 million T cells were aliquoted and centrifuged for 10 min at 90 g. During cell spin, 8.33 μl of HDR template (total 16.66 μg) per condition were aliquoted to PCR tubes, followed by RNPs (11.66 μl per well) and were incubated for at least 5 minutes. After cell centrifugation, supernatants were removed by vacuum, and cells were resuspended in 80 μl P3 buffer (Lonza), then transferred to PCR tubes containing RNPs and HDR templates, bringing the total volume per sample to 100 μl. Each sample was transferred directly to a 100 μL Nucleocuvette™ Vessel. T cells were electroporated with a Lonza 4D Nucleofector™ with X Unit using pulse code EH115. Immediately after nucleofection, 100 μl of pre-warmed recovery medium with 500 U/mL IL-2 and 25 μl/mL ImmunoCult™ CD3/CD28/CD2 activator was added to each cuvette. Cuvettes were rested at 37° C. in the cell culture incubator for 15 minutes. After 15 minutes, cells were moved to 200 μl total volume of recovery media and equally distributed to 4 wells round bottom 96 well plate.

Flow cytometry Analysis: T cells were stained and analyzed on day 7 of manufacture for mCherry and TCR expression. Ghost Dye™ Red780 was used as a live dead stain to access cell viability. TCR a/b antibody clone IP26 was used to detect TCR knockout in BD Brilliant Stain Buffer (BD Biosciences). All stained samples were run on an Attune™ NxT Flow cytometer (Thermo Fisher Scientific). T cells were stained and analyzed on day 7 of manufacture for mCherry and TCR expression, and day 10 of manufacture for the full Aurora immunophenotyping panel, using fresh cells. Downstream analyses of all spectral cytometry data were performed in FCS Express 7 Software.

In-out PCR: Following guidance from the art, genomic DNA was extracted from 100,000 cells per condition using DNA QuickExtract™ (Lucigen), and incubated at 65° C. for 15 min, 68° C. for 15 min, and 98° C. for 10 min. Genomic integration of the CAR was confirmed by in-out PCR using a forward primer upstream of the TRAC left homology arm, and a reverse primer binding within the CAR sequence. (ATCTTGTGCGCATGTGAGGGGC (SEQ ID NO: 64) and GCAAGCCAGGACTCCACCAACC (SEQ ID NO: 65). PCR was performed according to the manufacturer's instructions using Q5™ Hot Start Polymerase (NEB) using the following program: 98° C. (30 s), 35 cycles of 98° C. (10 s), 67° C. (20 s), 72° C. (2 min), and a final extension at 72° C. (2 min).

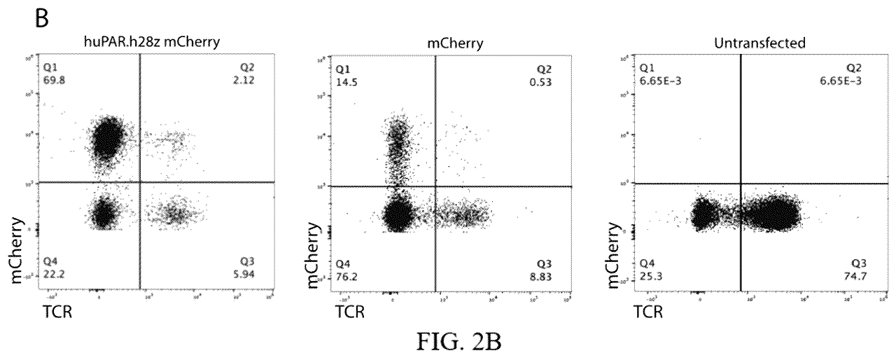

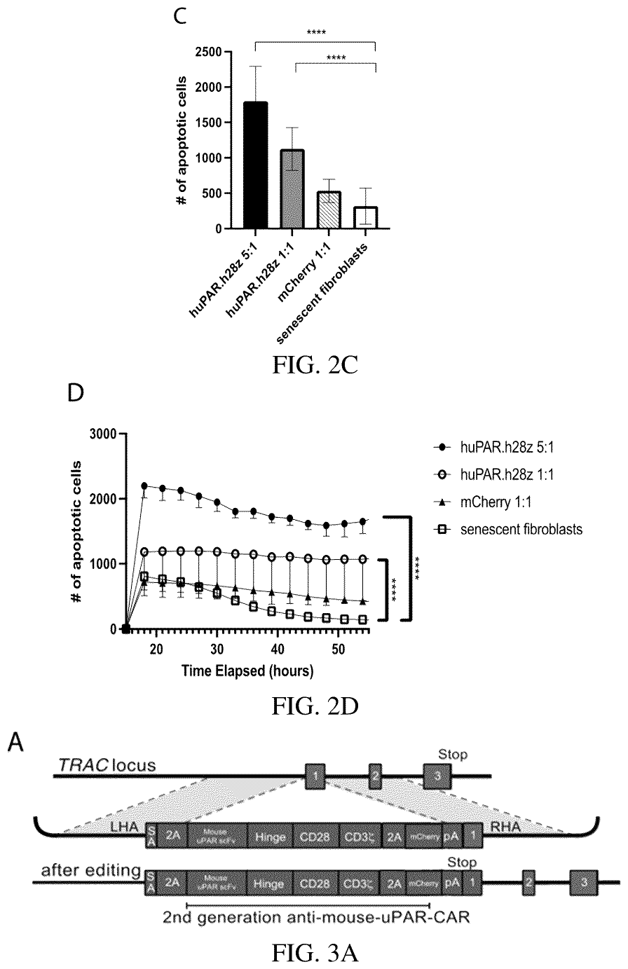

In Vitro Cytotoxicity Assays: For FIG. 2: 10,000 HDFa fibroblasts at varying passage numbers cells were seeded in triplicate per condition in a CytoView-Z 96 Well plate (Axion Biosytems) and maintained in the Maestro Z (Axion Biosystems) stored at 37° C., 5% CO2. Cell viability and impedance was tracked continuously for 24 hours and then treated with CDK4/6 and MEK media additives for 48 hours. VFC T cells were added to each well with varying effector: target ratios based to reach 100%, 50%, 25% CAR positivity. Cytotoxicity was measured every hour for 48 hrs and data output was imported and analyzed with AxIS software.

SA-β-Gal Staining: SA-β-gal staining was performed using CHEMICON® Cellular Senescence Assay Kit (cat. KAA002 Millipore Sigma) at a pH 6.0 for human cells. Adherent cells plated in a 12 well plate and fixed with 500 μl Fixing solution (Millipore Sigma) and incubated at room temperature for 10 minutes, washed twice with 1×PBS and stained with freshly prepared 1×SA-β-gal Detection Solution (Millipore Sigma) at 37° C., without CO2 and protected from the light and left overnight. The SA-β-gal Detection Solution was removed and the cells were washed with twice with 1×PBS. Blue stained cells were imaged on a Leica light microscope and three high power fields per well were counted and averaged to quantify the percentage of SA-β-gal+ cells per population.

### Example 1: Vfc-Hupar-Mcherry T Cells Eliminate Senescent Cell Populations in In Vitro Coculture Assay

To avoid the use of viral vectors in our manufacturing process we began by cloning a second generation huPAR CAR sequence with an appended mCherry fluorescent protein with homology arms at the desired cut site for the start of the first encoding exon, exon 6, of the TRAC locus (FIG. 2A). We next generated double-stranded DNA (dsDNA) HDR templates via PCR amplification and performed a two-step purification process first with a Solid Phase Reversible Immobilization (SPRI) with AMPureXP beads followed by an ethanol precipitation to purify and concentrate the templates.

Primary human T cells from healthy donors were electroporated with the purified HDR templates and SpyCas9 ribonucleoproteins (RNPs) targeting the human TRAC locus. Cells were recovered for 24 hours at a 1 million/mL density in round-bottom 96-well plates and were expanded in Immunocult™ xeno-free human T cell expansion medium. The cell viability and proliferation of VFC-huPAR-mCh was monitored over 9 days throughout the manufacturing process. Cells were then assayed on day 7 post-isolation to confirm the integration of the VFC-huPAR-mCh CAR T cell products as well as a virus-free CRISPR mCherry only control (VFC-mCh), in place of the huPAR-mCherry CAR sequence. We achieved consistently high genome editing with the dsDNA templates across 2 donors and demonstrated up to 70% knock-in efficiency, with an average of 20% uPAR+ and >90% total TCR-cells, as measured by flow cytometry (FIG. 2B).

To evaluate the efficiency of the uPAR-mCh CAR T cells in eliminating uPAR+ cells we measured the in vitro potency against senescent induced fibroblasts. Human dermal fibroblasts (HDFa) were plated at 30% confluency and allowed to adhere for 24 hrs, after incubation cells were induced with CDK4/6 and MEK inhibitors. The cells were then stained with SA-β-galactosidase to access for the presence of the senescence associated secretory phenotype (SASP). We performed an impedance assay measuring loss of resistance from induced and non-induced fibroblast populations over a 48 hour period. We observed potent killing 5:1 effector:target ratios. These results demonstrate potent target cell killing of uPAR+ senescent cells through multiple stimuli (FIG. 2C-D).

### Example 2: Vfc-Mupar-Ngfr T Cells Eliminate Murine Senescent Cell Populations in In Vitro Coculture Assay

To avoid the use of viral vectors in our manufacturing process we began by cloning a second generation muPAR CAR sequence with an appended a tNGFR selectable marker with homology arms at the desired cut site for the start of the first encoding exon, exon 6, of the TRAC locus (FIG. 1A). We next generated double-stranded DNA (dsDNA) HDR templates via PCR amplification and performed a two-step purification process first with a Solid Phase Reversible Immobilization (SPRI) with AMPureXP beads followed by an ethanol precipitation to purify and concentrate the templates.

Primary human T cells from healthy donors were electroporated with the purified HDR templates and SpyCas9 ribonucleoproteins (RNPs) targeting the human TRAC locus. Cells were recovered for 24 hours at a 1 million/mL density in round-bottom 96-well plates and were expanded in Immunocult™ xeno-free human T cell expansion medium. Cells were then assayed on day 7 post-isolation to confirm the integration of the VFC-muPAR-NGFR CAR T cell products. Genomic integration of muPAR-NGFR CAR was confirmed via “in-out” PCR amplification assay on genomic DNA extracted from 100,000 cells from both VFC-muPAR-NGFR and untransfected control cells with primers specific to the TRAC locus and CAR transgene (FIG. 1B). The cell viability and proliferation of VFC-muPAR-NGFR was monitored over 9 days throughout the manufacturing process.

To evaluate the efficiency of the muPAR-NGFR CAR T cells in eliminating uPAR+ cells we measured the in vitro potency against mouse senescent induced fibroblasts. Mouse dermal fibroblasts from Ail4 transgenic mice were plated at 30% confluency and allowed to adhere for 24 h, after incubation cells were induced with CDK4/6 and MEK inhibitors. The cells were then stained with SA-β-galactosidase to access for the presence of the senescence associated secretory phenotype (SASP). We observed potent killing of senescent cells at 5:1 effector:target ratio (FIG. 1C-D). These results demonstrate potent target cell killing of uPAR+ murine senescent cells through multiple stimuli.

Exemplary templates include:
SEQ ID NO: 1-DNA: muPAR.h28z tNGFR
SEQ ID NO: 2-protein: muPAR.h28z tNGFR
SEQ ID NO: 3-DNA: huPAR.h28z tNGFR
SEQ ID NO: 4-protein: huPAR.h28z tNGFR
SEQ ID NO: 5 DNA: huPAR.h28z mCherry
SEQ ID NO: 6 protein: huPAR.h28z mCherry
SEQ ID NO: 7 DNA: muPAR.m28z tNGFR
SEQ ID NO: 8 protein: muPAR.m28z tNGFR
SEQ ID NO: 9 DNA: muPAR.h28z mCherry
SEQ ID NO: 10 protein: muPAR.h28z mCherry
SEQ ID NO: 11 DNA: muPAR.m28z mCherry
SEQ ID NO: 12 protein: muPAR.m28z mCherry
SEQ ID NO: 13 DNA: muPAR.m28z APOE2
SEQ ID NO: 14 protein: muPAR.m28z APOE2
SEQ ID NO: 15 DNA: huPAR.h28z APOE2
SEQ ID NO: 16 protein: huPAR.h28z APOE2
SEQ ID NO: 17 DNA: miniXon huPAR.h28z mCherry
SEQ ID NO: 18 protein: miniXon huPAR.h28z mCherry
SEQ ID NO: 19 DNA: miniXon muPAR.m28z mCherry
SEQ ID NO: 20 protein: miniXon muPAR.m28z mCherry
SEQ ID NO. 21 DNA: miniXon muPAR.m28z APOE2
SEQ ID NO. 22 protein: miniXon muPAR.m28z APOE2
SEQ ID NO: 23 DNA: miniXon huPAR.h28z APOE2
SEQ ID NO: 24 protein: miniXon huPAR.h28z APOE2
SEQ ID NO: 25 DNA: muPAR.h28z APOE2
SEQ ID NO: 26 protein: muPAR.h28z APOE2

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A DNA HDR template for a transgene comprising a chimeric antigen receptor (CAR) gene for inserting the transgene into a T cell expressed gene to generate CAR T cells having the composition:
(left HA)-(SA)-(first self-cleaving peptide polynucleotide or IRES)-(optional inducible control sequence)-(uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional first secreted factor or first selection marker polynucleotide)-(optional third self-cleaving peptide polynucleotide or IRES)-(optional second secreted factor or second selection marker polynucleotide)-(polyA terminator)-(right HA);
or
(left HA)-(SA)-(first self-cleaving peptide polynucleotide or IRES) (uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional inducible control sequence)-(optional first secreted factor or first selection marker polynucleotide)-(optional third self-cleaving peptide polynucleotide or IRES)-(optional second secreted factor or second selection marker polynucleotide)-(polyA terminator)-(right HA);
or
(left HA)-(SA)-(first self-cleaving peptide polynucleotide or IRES) (uPAR binding fragment polynucleotide)-(hinge domain polynucleotide)-(transmembrane domain polynucleotide)-(intracellular domain polynucleotide)-(optional second self-cleaving peptide polynucleotide or IRES)-(optional first secreted factor or first selection marker polynucleotide)-(optional third self-cleaving peptide polynucleotide or IRES)-(optional inducible control sequence)-(optional second secreted factor or second selection marker polynucleotide)-(polyA terminator)-(right HA);
wherein the left HA and the right HA are homology arms complementary to sequences on both sides of a cleavage site in the T cell expressed gene;
wherein SA is a splice acceptor site;
wherein the first, second and third self-cleaving peptide polynucleotide or IRES are polynucleotides encoding a first, second and third self-cleaving peptide or an internal ribosome entry site (IRES), respectively;
wherein the optional inducible control sequence is a regulatory sequence which provides control of protein expression in response to a small molecule inducer;
wherein the uPAR binding fragment polynucleotide is a polynucleotide encoding a polypeptide that specifically binds uPAR;
wherein the hinge domain polynucleotide encodes a CD28 or CD8α hinge domain;
wherein the transmembrane domain polynucleotide encodes a transmembrane domain;
wherein the intracellular domain polynucleotide encodes one or more intracellular domains;
wherein the first and second secreted factor polynucleotides are coding sequences for a neurotrophic factor, growth factor, or cytokine;
wherein the first and second selection marker polynucleotides are coding sequences for a detectable protein; and
wherein the polyA terminator is a sequence-based element that defines the end of a transcriptional unit.

2. The template of claim 1, wherein the left homology arm comprises 383 to 588 bp of the TRAC locus directly upstream of the cutsite, and the right homology arm includes 391 to 499 bp of the TRAC locus directly downstream of the cutsite.

3. The template of claim 1, wherein the first, second and third self-cleaving peptides independently comprise a porcine teschovirus-1 (P2A) peptide, a Thosea asigna virus (T2A) peptide, an equine rhinitis A virus (E2A) peptide, or a foot-and-mouth disease virus (F2A) peptide.

4. The template of claim 1, wherein the uPAR binding fragment is an antibody fragment.

5. The template of claim 1, wherein the uPAR binding fragment is a single-chain variable fragment comprising a heavy variable fragment and a light chain variable fragment.

6. The template of claim 1, wherein the transmembrane domain is from CD28 and the intracellular domain is a portion of CD3-zeta.

7. The template of claim 1, further comprising a polynucleotide encoding a costimulatory domain between the transmembrane domain polynucleotide and the intracellular domain polynucleotide.

8. The template of claim 7, wherein the costimulatory domain is OX40, 41BB, ICOS, CD27, CD40, CD40L or a TLR.

9. The template of claim 1, wherein the first and second secreted factors are each independently a pro-regenerative secreted factor, a pro-memory secreted factor, growth factor, or a factor that attracts pro-regenerative immune cells.

10. The template of claim 1, wherein the first selection marker, second selection marker or both are a coding sequence for a fluorescent protein.

11. A plasmid containing a sequence coding for the HDR template of claim 1.

12. The plasmid of claim 11 comprising a virus-free plasmid.

13. An ex vivo, virus-free method of site-specifically inserting a transgene containing a chimeric antigen receptor (CAR) gene into a T cell expressed gene to generate CAR T cells, comprising
preparing the homology-directed repair (HDR) DNA template of claim 1,
introducing into a population of unmodified T cells a Cas9 ribonucleoprotein (RNP) and the HDR template to provide the CAR T cells,
wherein the Cas9 RNP comprises a Cas9 protein and a guide RNA that directs double stranded DNA cleavage of a cleavage site in the T cell expressed gene, and
wherein the transgene is specifically integrated into the cleavage site of the T cell expressed gene locus created by the Cas9 RNP in the cells, and
culturing the CAR T cells in xeno-free medium to provide a cultured population of CAR T cells having the transgene specifically integrated in the T cell expressed gene,
wherein, in the cultured population of CAR T cells, an endogenous promoter of the T cell expressed gene drives expression of the transgene, or wherein the transgene includes a promoter that drives expression of the transgene, and
wherein the CAR gene encodes a fusion protein comprising the translated anti-uPAR binding motif, hinge, transmembrane domain, and one or more intracellular domain(s).

14. The method of claim 13, wherein the unmodified T cells are autologous T cells isolated from a patient, or T cells from an allogeneic healthy donor.

15. The method of claim 13, further comprising administering the cultured population of CAR T cells to a patient in need of treatment for a neurodegenerative disease, stroke, craniocerebral trauma and/or accident, or an elderly patient in need of treatment for aging.

16. The method of claim 15, wherein the neurodegenerative disease is Alzheimer's disease, dementia, Parkinson's disease, Lewy body disease, ataxia, Huntington's disease, amyotrophic lateral sclerosis, Down syndrome, or spinal muscular atrophy.

17. The method of claim 13, wherein administering is by intravenous or intracerebroventricular infusion or intracerebral injection.
